(19) United States
(12) Patent Application Publication
(10) Pub. No.: US 2015/0293105 A1
(43) Pub. Date: Oct. 15, 2015

(54) METHODS FOR CLASSIFYING TUMORS AND USES THEREFOR

(71) Applicant: THE UNIVERSITY OF QUEENSLAND, Queensland (AU)

(72) Inventors: Fiona Simpson, Paddington (AU); Nicholas Andrew Saunders, Holland Park (AU)

(73) Assignee: THE UNIVERSITY OF QUEENSLAND, St. Lucia, Queensland (AU)

(21) Appl. No.: 14/438,423

(22) PCT Filed: Oct. 28, 2013
(86) PCT No.: PCT/AU2013/001247

§ 371 Date: Apr. 24, 2015

(30) Foreign Application Priority Data
Oct. 26, 2012 (AU) 2012904721

(51) Int. Cl. G01N 33/574; C07K 16/30; C07K 16/28

(52) U.S. Cl. CPC G01N 33/57492; C07K 16/2863; C07K 16/30; G01N 2333/71; C07K 2317/76

(57) ABSTRACT

Disclosed are methods for classifying tumors into EGFR antagonist sensitive or resistant subtypes. Additionally, the present invention discloses methods for stratifying subjects with cancer into treatment subgroups based on whether their tumors fall into one of these subtypes and to methods for treating subjects so stratified.

This application claims priority to Australian Provisional Application No 2012904721 entitled “Methods for Classifying Tumors and Uses Therefor” filed 26 Oct. 2012, the contents of which are incorporated herein by reference in their entirety.

### FIELD OF THE INVENTION

This invention relates generally to methods for classifying tumors into EGFR antagonist sensitive or resistant subtypes. The present invention also relates to methods for stratifying subjects with cancer into treatment subgroups based on whether their tumors fall into one of these subtypes and to methods for treating subjects so stratified.

Bibliographic details of various citations referred to numerically in the present specification are listed at the end of the description.

### BACKGROUND OF THE INVENTION

The role of inappropriate activation of receptor tyrosine kinases (RTKs) of the erb family in the pathogenesis of cancer has prompted the development of molecular inhibitors of these receptors, especially the epidermal growth factor receptor (EGFR) and its signaling pathway components. EGFR signaling pathways control important cellular functions such as proliferation, survival, differentiation, migration and tissue remodeling. Gene amplification, protein overexpression and constitutive activation of the EGFR are common mechanisms underlying aberrant signaling in several cancers, including epithelial tumors. Most significantly, recurrent squamous cell carcinoma (SCC) after conventional therapy is EGFR dependent (1, 2).

The erbB RTK family consists of 4 single-transmembrane domain receptors which can homo- and hetero-dimerize upon ligand binding. Activation of the receptors by ligand binding initiates two central signaling cascades in the cell. One pathway activates KRAS which activates BRAF which, in turn, triggers the mitogen-activated protein (MAPK) cascade. The other pathway localizes PIK3CA, inhibiting PTEN and leading to AKT1 phosphorylation.

Anti-EGFR monoclonal antibodies in clinical use bind to the extracellular domain of EGFR in its inactive state. This prevents ligand binding and ligand-induced receptor activation. Antibodies specific for the EGFR are now in clinical use for a number of malignancies. Despite their pro-inflammatory side effects, promising results have been shown in some patients treated with anti-EGFR antibodies, either monotherapy or in combination with radio- or chemo-therapy (3). However, a significant proportion of treated patients fail to respond for reasons which have not yet been fully elucidated. An exciting new study in Nature Medicine (4) has shown that some patients carry a mutation in the EGFR which prevents cetuximab binding but not panumitumab binding and thus a further percentage of patients can be screened and be treated with the appropriate anti-EGFR antibody. However, this does not bypass most observed patient resistance. Numerous studies have been carried out to ascertain the mechanism of the noted resistance. EGFR expression is increased in squamous cell carcinoma and other epithelial tumors by several different mechanisms including amplification, gain of expression of the gene and increased rate of EGFR activating mutations.

While specific alterations in EGFR number or function were predicted to affect treatment response, no correlation has been observed in clinical trials (5-9). In ˜10% of patients resistant to EGFR targeted therapy there are specific activating mutations in the downstream signaling components of the EGFR such as KRAS in the tumors. There is a significant association between absence of response to cetuximab and KRAS status (10-12). A BRAF point mutation, V600E, is one of the most common oncogenic mutations in cells, and a retrospective analysis (13) of patients with metastatic colorectal cancer showed that no BRAF-mutated tumors responded to cetuximab or panitumumab. Analysis of KRAS and BRAF mutational status is now becoming commonplace prior to the use of anti-EGFR antibodies, allowing patients who are unlikely to respond to be selected out and offered alternate treatment such as anti-VEGF therapy. However, these mutations only involve 10% of resistant patients. Mass spectrometry profiling of patient tumor proteomes from responsive versus resistant patients (5) has also been used to predict patient response to EGFR inhibitor treatment but at present the algorithms are not robust enough for use in patient analysis. Attempts to correlate tumor resistance with specific mutations in genes of the EGFR family now involve simultaneous profiling many of genes, which is time-consuming and expensive.

### SUMMARY OF THE INVENTION

The present invention arises from the unexpected discovery that EGFR positive tumors having impaired or abrogated ligand-induced EGFR internalization (also referred to herein as “disregulated EGFR”) are sensitive to EGFR antagonist therapy (e.g., using an anti-EGFR antibody such as cetuximab or panitumumab), whereas EGFR positive tumors having unimpaired ligand-induced EGFR internalization are resistant or refractory to EGFR antagonist therapy. Based on this discovery, the present inventors propose methods for classifying tumors into different clinical subtypes or for stratifying tumor-affected subjects into different treatment subgroups according to the ligand-induced EGFR internalization status of the tumor. These methods enable better selection of treatment of tumors and affected subjects, as described hereafter.

Thus, the present invention addresses the problem of distinguishing between EGFR antagonist responders and non-responders by determining the degree of ligand-induced EGFR internalization in tumors from cancer-affected subjects. This represents a significant advance over current technologies for the management of EGFR positive cancers.

Accordingly, in one aspect, the present invention provides methods for classifying an EGFR positive tumor into a subtype selected from an EGFR antagonist sensitive subtype or an EGFR antagonist resistant subtype. These methods generally comprise, consist or consist essentially of analyzing the ligand-induced EGFR internalization status of the tumor, wherein an impaired or abrogated ligand-induced EGFR internalization status, suitably relative to a control, indicates that the tumor is an EGFR antagonist sensitive subtype and wherein an unimpaired ligand-induced EGFR internalization status, suitably relative to a control, indicates that the tumor is an EGFR antagonist resistant subtype. Suitably, the ligand-induced EGFR internalization status is analyzed in the absence of analyzing KRAS status and/or BRAF status of the tumor. In some embodiments, the ligand-induced EGFR internalization status is analyzed in the absence of analyzing molecules involved in EGFR-associated downstream signaling. In some embodiments, an impaired or abrogated ligand-induced EGFR internalization is indicated when, suitably after at least 10 minutes (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more minutes) in the presence of an EGFR ligand (e.g., EGF, TGF-α, amphiregulin, heparin-binding EGF-like growth factor, betacellulin, and epiregulin), at least 90% (e.g., 90%, 91%, 92%, 93%, 94%, 95%, 96%, 97%, 98%, 99% or 100%) of the EGFR in cells of the tumor is localized or remains localized to the plasma membrane (e.g., basolateral membrane) of the cells.

The tumor suitably includes pre-cancerous, non-metastatic, metastatic, and cancerous tumors (e.g., early stage cancer). Representative cancers are selected from carcinoma, lymphoma, blastoma, sarcoma, neuroendocrine tumors, mesothelioma, schwannoma, meningioma, adenocarcinoma, melanoma, leukemia, and lymphoid malignancies. In some embodiments, the cancer is selected from lung cancer, hepatocellular cancer, gastric or stomach cancer, pancreatic cancer, glioblastoma, cervical cancer, ovarian cancer, liver cancer, bladder cancer, hepatoma, breast cancer, colon cancer, rectal cancer, colorectal cancer, endometrial and uterine carcinoma, salivary gland carcinoma, kidney cancer, prostate cancer, vulval cancer, thyroid cancer, hepatic carcinoma, anal carcinoma, penile carcinoma, testicular cancer, esophageal cancer, tumors of the biliary tract, and head and neck cancer. In specific embodiment, the tumor is of an epithelial origin, non-limiting examples of which include cancer of the lung, colon, prostate, ovary, breast, and skin (e.g., squamous cell carcinoma (SCC)).

In a related aspect, the present invention provides methods for stratifying a subject with an EGFR positive cancer, as for example described above, into a treatment subgroup selected from responder to an EGFR antagonist and non-responder to an EGFR antagonist. These methods generally comprise, consist or consist essentially of classifying an EGFR positive tumor according to the tumor classification methods as broadly described above, and identifying the subject as a responder to an EGFR antagonist if an EGFR positive tumor of the subject is analyzed as having an impaired or abrogated ligand-induced EGFR internalization status or identifying the subject as a non-responder to an EGFR antagonist if an EGFR positive tumor of the subject is analyzed as having an unimpaired ligand-induced EGFR internalization status. In some embodiments, the methods further comprise obtaining a tumor sample from the subject for the analysis.

Another aspect of the present invention provides methods for treating a subject with an EGFR positive cancer, as for example described above. These methods generally comprise, consist or consist essentially of stratifying the subject into a treatment subgroup selected from responder to an EGFR antagonist and non-responder to an EGFR antagonist, as broadly described above, and administering an EGFR antagonist to the subject on the basis that the subject is stratified into the responder subgroup or administering a cancer therapy other than an EGFR antagonist to the subject on the basis that the subject is stratified into the non-responder subgroup. In some embodiments, the EGFR antagonist is selected from anti-EGFR antibodies such as, but not limited to cetuximab, panitumumab, nimotuzumab and matuzumab, or anti-EGFR tyrosine kinase inhibitors, illustrative examples of which include gefitinib and erlotinib. In some embodiments, the methods further comprise co-administering an ancillary cancer therapy to the subject, illustrative examples of which include radiotherapy, surgery, chemotherapy, hormone ablation therapy, pro-apoptosis therapy and immunotherapy other than the antibody.

### BRIEF DESCRIPTION OF THE DRAWINGS

FIG. 1A is a photographic representation showing receptor endocytosis in cell lines by Alexa-fluor labeling of EGFR before and after the addition of EGF.

FIG. 1B is a photographic and graphical representation showing EGF uptake versus disregulation in representative skin tumor samples. Panels A and B show DAPI stained nuclei, Panels C and D show EGF-Alexa488 (direct conjugation) in green emission channel and Panels E and F show the merged images. Panel C shows internalized EGF in Sample 1 while Panel D shows non-internalizing plasma membrane localized EGF in Sample 2. Panel G shows quantitation of internalized versus plasma membrane bound EGF at 30 minute stimulation time point is shown for Sample 1 (Image 1) and sample 2 (Image 2).

FIG. 2 is a photographic representation depicting an invasive SCC shown by Hematoxylin and Eosin (H&E) staining and confocal microscopy. The epidermis is thickened and dysplastic. Nuclear disorganization and invasion can be seen clearly in the RCM image in which nuclei stained with DAPI are shown. There is a dense inflammatory infiltrate in the dermis. Little stratum corneum is visualized in either image in this case.

FIG. 3 is a photographic representation showing a normal skin sample panel, RCM 63λ. Nuclei are stained with DAPI. The corneocyte envelopes strongly autofluoresce in the red emission spectrum. Panel A shows DAPI staining, panel B shows the red emission channel and Panel C shows a merged image of the two channels. Nuclei are sparse in comparison to dysplastic samples and are widely spaced. The epidermis is thin and there is a thin layer of normal orthokeratosis. There is a well demarcated basal layer with no invasion or inflammatory infiltrate visible.

FIG. 4 is a photographic and graphical representation showing nuclear quantitation in SCC and normal skin. Assessment of mean cross sectional area and aspect ratio also demonstrates the expected difference between nuclei of normal and dysplastic keratinocytes. A larger aspect ratio indicates deviation away from a perfect circle. The upper panel shows SCC nuclei, a magnified area chosen for quantitation and the area picked by the imaging software for quantitation. The mid-panels show the same for a control ‘normal’ skin sample. The left graph shows the nuclear area quantitation of the two samples and the right graph shows quantitation of the nuclear aspect ratio.

FIG. 5 is a graphical representation showing an example of nuclear morphometry data from 3 lesions with associated normal skin controls. Nuclear cross-sectional area for each lesion was significantly different from its matched control skin sample (P<0.05). Here normal skin is shown as the mean of the data from all three control samples. Although the lesions show the predicted increase in nuclear area with increasing invasiveness this was not significant.

FIG. 6 is a photographic representation showing examples of tumors demonstrating normal ligand-induced receptor-mediated endocytosis of EGF. A. DCB177033 is a well differentiated SCC from the back of an elderly male. The tumor extended to the deep dermis. The difference in nuclear density and disorganization between the tumor sections and adjacent uninvolved skin is apparent. A small amount of specific EGF-488 binding is seen at the 15 minute time point. Normal ligand-induced EGF internalization is seen at the 30 minute time point. Cetuximab prevents uptake of EGF. There is minimal EGF-488 uptake in the control skin even at the 30 minute time point. B. DCV299052 is an IEC from the forearm of a female. The control skin from adjacent to the lesion showed marked variability. Some regions appeared normal in gross appearance and these demonstrated little EGF-488 uptake or EGFR positivity. C. Other sections of the control skin were atypical in nuclear appearance and demonstrated increased EGFR positivity and EGF uptake. As the control skin is taken from immediately adjacent to the tumor and judged to be non-involved by the naked eye only, there is potential for tumor tissue to be found also in these control samples. This may also reflect the severe photo-damage observed on a more exposed body site. Again normal internalization of EGF-488 is observed by 30 minutes unless tissue is first treated with Cetuximab as a competitive inhibitor of EGF-488 binding.

FIG. 7 is a photographic representation showing examples of tumors demonstrating loss of ligand-induced EGFR endocytosis, termed as ‘disregulated.’ EGF-488 remains localized at the plasma membrane at the 30 minute time point. DCM2710056 is an SCC from the forearm of a 60 year old man, while DCB238043 is an SCC from the neck of an 80 year old female. No significant EGF internalization is seen in control normal skin samples.

FIG. 8 is a photographic representation showing EGFR expression across the epidermis. EGFR expression in the epidermis is shown in red in the original image on the left. Expression can be seen more clearly in greyscale without nuclei. In the final image EGFR intensity has been thresholded to exclude staining in the upper levels of the epidermis. Strong expression can still be seen in the basal layers of the epidermis in several regions. The stratum corneum demonstrates bright autofluorescence in the red channel but does not express EGFR.

FIG. 9 is a photographic and diagrammatic representation showing predominant uptake in basal layers and leading edge of tumors. Various subtype patterns were seen. Some are not polarized while some show strong polarization of uptake to the basal pole of the cells. As EGFR signaling is known to be involved in metastasis and cytokinetic migration this may be a significant observation.

FIG. 10 is a photographic representation showing trafficking status of squamous lesions: pies charts. Trapped indicates tumors whose EGFR no longer undergoes ligand-induced endocytosis and remains localized to the plasma membrane. Internalized indicates tumors whose EGFR underwent ligand-induced receptor-mediated endocytosis and at 15- or 30-minute time-points showed endosomal localization of EGFR.

FIG. 11 is a graphical representation showing a tumor data set according to trafficking status and lesion type. No disregulated IECs were identified.

FIG. 12 is a graphical representation showing trafficking status and tumor grade frequency. Non-invasive precursor lesions are not assigned a pathological grade and so are not applicable (NA). The trafficking status of all poorly differentiated samples could be identified whereas in all other groups at least one lesion was in the not visualized category.

FIG. 13 is a graphical representation showing trafficking status and tumor type by depth of invasion.

FIG. 14 is a graphical representation showing trafficking status and correlation with multifactorial risk stratification.

FIG. 15 is a graphical representation showing: A Plot of number of previous SCCs in sample population. Peaks are seen at Zero and five creating three subgroups. B. Trafficking status compared to subgroup of prior history of SCC. Disregulated tumors were not seen in patients who had no previous SCCs. Disregulation was most common in patients with five or more previous SCCs. EGFR internalizing tumors occurred at a steady frequency across frequency groups. When high risk tumors are considered separately these were seen in the history of more than one third of internalizing cases but no patients with disregulated tumors fell into this category.

FIG. 16 is a graphical representation showing expression of total EGFR by quantification of tumor section immunofluorescence. Basal layers of the epidermis were analyzed in matched pairs of internalizing and disregulated tumors with similar histopathological tumor features. Total EGFR expression levels did not increase with increasing tumor invasiveness or grade. There was no correlation between trafficking status and EGFR total expression levels.

FIG. 17 is a photographic representation showing the distribution of EGF-Alexa⁴⁸⁸ after 15 min stimulation in SCC cell monolayer is comparable to the SCC-derived mouse xenograft. (A, B) Images of Ca127 cell line and xenograft derived from Cal 27 both stimulated with EGF-Alexa⁴⁸⁸ for 15 min. Nuclei are shown in blue and EGF-Alexa⁴⁸⁸ in green. (A.1, B.1) Higher magnification image of EGF-Alexa⁴⁸⁸ of selected cell as indicated by the box corresponding to image A and B, respectively. (C, D) Images of Detroit cell line and xenograft derived from Detroit cells both stimulated with EGF-Alexa⁴⁸⁸ for 15 min. (C.1, BA) Higher magnification image of EGF-Alexa⁴⁸⁸ of selected cell as indicated by the insert corresponding to image C and D respectively. (E, F) Images of KJD cell line and xenograft derived from KJD cells both stimulated with EGF-Alexa⁴⁸⁸ for 15 min. (E.1, F.1) Higher magnification image of EGF-Alexa⁴⁸⁸ of selected cell as indicated by the insert corresponding to image E and F respectively. (Cell line: n=3; Xenograft: n=2, representative image shown). (Scale bars, 20 μm).

FIG. 18 is a photographic representation showing that internalization of EGF-Alexa⁴⁸⁸ differs between patient SCC. (A) EGF-Alexa488 distribution in patient SCC after 15 min and 30 min stimulation. Inserts are higher magnification of the region shown by the smaller box. Uptake of EGF into endosomal structures is observed and this phenotype is seen in approximately 40% of patients (refer to table 1). (B) EGF-Alexa488 distribution in patient SCC after 15 min and 30 min stimulation. Inserts are a higher magnification image of the region indicated by the smaller box. Plasma membrane binding of EGF is observed but with little internalization. This is representative of approximately 60% of patients (see Table 1). EGF-Alexa⁴⁸⁸ distribution in corresponding normal patient epithelial tissue is shown at 30 min. (C) Pre-treatment with Cetuximab prior to EGF-Alexa488 addition inhibits EGF binding. Cetuximab was labeled post-fixation using anti-human Alexa-594 secondary. In the merged images Cetuximab is shown in red, EGF in green and nuclei in blue. (D) Post-fixation labeling of the EGFR (red) shown in the merged image co-localizes with EGF ligand (green). (Scale bars, 20 μm).

FIG. 19 is a photographic representation showing the dysplastic cells within patient SCC samples as identified by H&E staining over-express the EGFR. (A, B) H&E staining of 4 μm sections from patient tumors taken at 4× magnification (Scale bar, 200 (C, D) 25× magnification images of region indicated by white box in panels A and B respectively. (E,F) Successive sections of C and D fluorescently labeled with EGFR (green) and DAPI stained (blue) (Scale, 50 μm).

FIG. 20 is a graphical and photographic representation showing the rates of EGF internalization in SCC cell lines are variable and exhibit disregulation in initial uptake. (A) Biotin-EGF uptake was performed in SCC cell lines for the time points indicated. Endocytosis was measured as avidin inaccessibility as a percentage of total at 15 min. Assays were performed as described in Materials and methods. Data shown are the average of 3 experiments +/−SEM. (B) Levels of EGFR expression in SCC cell lines are increased compared to HEK cells. Equal protein concentrations of SCC and HEK cell lysates were subjected to ELISA assay for EGFR levels (Materials and Methods). Data shown are an average of 3 experiments +/−SEM by non-paired T-test. ***: P<0.001; **: P<0.01; *: P<0.05. ns: no significance. (C and D) Correlation graphs of internalized EGF (y-axis) versus EGFR expression level (x-axis) at 5 minute (C) and 15 minute (D) time points of EGF internalization. (E and F) EGF-Alexa⁴⁸⁸ (E) or Tfn-Alexa⁵⁹⁴ uptake was performed in SCC cell lines as described in Materials and Methods. Coverslips were fixed and imaged by confocal microscopy at 15 min.

FIG. 21 is a photographic representation illustrating that SCC cell lines show differing levels of activation of the EGFR and downstream pathway signaling targets. (A) Western analysis of SCC cell lysates. 10 μg of cell lysates indicated were loaded per well. Levels of EGFR expression are shown along with tubulin loading control. (B) Western analysis of EGFR phosphorylation, AKT phosphorylation relative to total AKT and ERK phosphorylation relative to total. ERK levels. Cells were basalled by incubation for four hours in serum free media or basalled and then stimulated with low concentration of EGF (1 ng/mL) for 15 min then washed in cold PBS. Cell lysates were prepared as described in Materials & Methods (Quantitative EGF Internalization Assay) and subjected to SDS-PAGE and Western analysis. Full time-courses of stimulation were completed and analyzed.

FIG. 22 is a photographic representation showing that EGFR staining can be used as a marker to visualize endocytosis differences in patient samples stimulated with EGF ligand. Samples DP5 and EG7 have been stimulated with EGF ligand for 30 min prior to fixation and EGFR labeling. Post-fixation labeling of the EGFR is shown. Inserts are a higher magnification of the region indicated by the smaller box. (Scale bars, 20 μm).

FIG. 23 is a photographic representation showing. Super-resolution microscopy of human tumor EGFR endocytosis. (A) Super-resolution microscopy of a human SCC in which EGFR does not undergo normal ligand-induced internalization (Plasma membrane (blocked)) and a human SCC in which EGFR retains ligand-induced internalization function. At this resolution the different distribution of EGF-Alexa⁴⁸⁸ in the two samples could be clearly observed. Nuclei are indicated by white dotted lines. The endosomal structures in the human tissue appear quite large in comparison to similar structures observed in tissue culture cells. Scale bars: 5 μm. (B) Co-localization of clathrin and internalized. EGF in tumors AC3P and DP5.

Some figures and text contain color representations or entities. Color illustrations are available from the Applicant upon request or from an appropriate Patent Office. A fee may be imposed if obtained from a Patent Office.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, illustrative methods and materials are described. For the purposes of the present invention, the following terms are defined below.

The use of numerical values in the various ranges specified in this application, unless expressly indicated otherwise, are stated as approximations as though the minimum and maximum values within the stated ranges were both preceded by the word “about.” In this manner, slight variations above and below the stated ranges (e.g., less than or equal to 10%, 9%, 8%, 7%, 6%, 5%, 4%, 3%, 2%, 1%) can be used to achieve substantially the same results as values within the ranges. Also, the disclosure of these ranges is intended as a continuous range including every value between the minimum and maximum values.

The articles “a” and “an” are used herein to refer to one or to more than one (i.e. to at least one) of the grammatical object of the article. By way of example, “an element” means one element or more than one element.

The term “antagonist” as used herein refers to a molecule that binds to or otherwise interacts with a receptor to block (e.g., inhibit) activation of that receptor by an agonist.

As used herein, the term “BRAF status,” refers to whether a patient's tumor is negative for an activating BRAF mutation (BRAF-negative) or positive for an activating BRAF mutation (BRAF-positive). An “activating BRAF mutation” refers to a mutation in a k-ras gene that results in constitutive activation of a protein encoded by B-Raf, i.e. the BRAF protein activates molecules downstream in its signaling pathway in the absence of receptor bound ligand. As an example, the BRAF protein might activate downstream signaling in the absence of EGF, amphiregulin, or epiregulin binding to EGFR.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

“Downstream signaling” refers to the modulation (e.g., stimulation or inhibition) of a cellular function/activity after binding of a ligand to the receptor. Examples of such functions include mobilization of intracellular molecules that participate in a signal transduction pathway, or that result in a change in the amount of intracellular molecules, alteration in the structure of a cellular component, such as for example the sarcomere, cell differentiation and cell survival.

By “effective amount,” in the context of treating or preventing a disease or condition (e.g., a cancer) is meant the administration of an amount of active agent to a subject, either in a single dose or as part of a series or slow release system, which is effective for the treatment or prevention of that disease or condition. The effective amount will vary depending upon the health and physical condition of the subject and the taxonomic group of individual to be treated, the formulation of the composition, the assessment of the medical situation, and other relevant factors.

As used herein, the term “epidermal growth factor receptor” (“EGFR”) refers to a membrane polypeptide that binds, and is thereby activated by, epidermal growth factor (EGF). EGFR is also known in the literature as ERBB, ERBB1 and HER1. An exemplary EGFR is the human epidermal growth factor receptor (see, Ullrich et al. (1984) Nature 309:418-425; GenBank accession number NP005219.2). Binding of an EGF ligand activates the EGFR (e.g., resulting in activation of intracellular mitogenic signaling, autophosphorylation of EGFR). EGFR is known to bind ligands including EGF, transforming growth factor-α (TGF-α), amphiregulin, heparin-binding EGF (hb-EGF), betacellulin, and epiregulin (Herbst, R. S., and Shin, D. M., Cancer 94 (2002) 1593-1611; Mendelsohn, J., and Baselga, J., Oncogene 19 (2000) 6550-6565). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al. (2003) Ann. Oncology 14:1346-1363; Tsao, A. S., and Herbst, R. S., (2003) Signal 4:4-9; Herbst, R. S., and Shin, D. M., (2002) Cancer 94:1593-1611; Modjtahedi, H., et al. (1996) Br. J. Cancer 73:228-235).

As used herein an “EGFR antagonist sensitive tumor” refers to an EGFR positive tumor in which at least 30%, 40%, 50%, 60%, 70%, 80%, 90%, 95% or up to 100% of EGFR-expressing cells in the tumor have an impaired or abrogated ligand-induced EGFR internalization.

As used herein an “EGFR antagonist resistant tumor” refers to an EGFR positive or negative tumor in which at least 30%, 40%, 50%, 60%, 70%, 80%, 90%, 95% or up to 100% of cells in the tumor have an unimpaired ligand-induced EGFR internalization.

The term “EGFR-expressing cell” as used herein refers to cells that express a cell surface EGFR polypeptide. “EGFR expression” refers to conversion of the information encoded in by the c-erbB proto-oncogene into messenger RNA (mRNA) and then to the EGFR polypeptide.

The term “EGFR-positive tumor” as used herein refers to a tumor that contains at least 1%, particularly at least 2%, 3%, 4% or 5%, particularly at least 10%, 20%, 30%, 40%, 50%, 60%, 70%, 80%, 90% EGFR-expressing cells, detected e.g. by an immunohistochemistry test such as, for example, the EGFR pharmaDx kit (“DAKO” test; DAKO North America, Inc.), the Zymed EGFR kit or the Ventana EGFR 3C6 antibody. In specific embodiments, the EGFR positive cells overexpress EGFR. By “overexpression of EGFR” and the like is intended to mean an abnormal level of expression of EGFR in a cell from a tumor within a specific tissue or organ of a patient relative to the level of expression in a normal cell from that tissue or organ. Patients having a cancer characterized by overexpression of the EGFR can be determined by standard assays known in the art, as for example noted above. Cancers characterized by EGFR-positive tumor are referred to herein as “EGFR-positive cancers.”

The term “impaired ligand-induced EGFR internalization” or “impaired internalization of EGFR” refers to reduced or abrogated internalization of EGFR in an EGFR positive cell from a tumor when the EGFR is bound by a cognate ligand (e.g., EGF, TGF-α, amphiregulin, heparin-binding EGF-like growth factor, betacellulin, and epiregulin), as compared with internalization of EGFR in a normal EGFR-expressing cell when the EGFR is bound by the same ligand. In specific embodiments, an impaired or abrogated ligand-induced internalization of EGFR is indicated when, suitably after at least 10 minutes (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more minutes) in the presence of an EGFR ligand (e.g., EGF, TGF-α, amphiregulin, heparin-binding EGF-like growth factor, betacellulin, and epiregulin), at least 90% (e.g., 90%, 91%, 92%, 93%, 94%, 95%, 96%, 97%, 98%, 99% or 100%) of the EGFR in cells of the tumor is localized or remains localized to the plasma membrane (e.g., basolateral membrane localization) of the cells. By contrast, an “unimpaired ligand-induced EGFR internalization” or “unimpaired internalization of EGFR” refers to the same, similar or greater internalization of EGFR in an EGFR positive or negative cell from a tumor when the EGFR is bound by a cognate ligand, as compared with internalization of EGFR in a normal EGFR-expressing cell when the EGFR is bound by the same ligand. In some embodiments, an unimpaired ligand-induced internalization of EGFR is indicated when, suitably after at least 10 minutes (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more minutes) in the presence of an EGFR ligand, less than 90%, 85%, 80%, 75%, 70%, 65%, 60%, 55%, 50%, 45%, 40%, 35%, 30%, 25%, 20%, 15%, 10% or even less of the EGFR in cells of the tumor is localized or remains localized to the plasma membrane (e.g., basolateral membrane localization) of the cells.

As used herein, the term “KRAS status,” refers to whether a patient's tumor is negative for an activating KRAS mutation (KRAS-negative) or positive for an activating KRAS mutation (KRAS-positive). The term “activating KRAS mutation” refers to a mutation in a k-ras gene that results in constitutive activation of a protein encoded by K-Ras, i.e. the KRAS protein activates molecules downstream in its signaling pathway in the absence of receptor bound ligand. As an example, the KRAS protein might activate downstream signaling in the absence of EGF, amphiregulin, or epiregulin binding to EGFR.

As used herein, the terms “label” and “detectable label” refer to a molecule capable of being detected, where such molecules include, but are not limited to, radioactive isotopes, fluorescers (fluorophores), chemiluminescers, chromophores, enzymes, enzyme substrates, enzyme cofactors, enzyme inhibitors, dyes, metal ions, metal sols, ligands (e.g., biotin, avidin, streptavidin or haptens), intercalating dyes and the like. The term “fluorescer” or “fluorophore” refers to a substance or a portion thereof which is capable of exhibiting fluorescence in a detectable range.

The term “ligand” as used herein refers to a naturally occurring or synthetic compound that binds to a receptor (e.g., EGFR). Upon binding to a receptor, ligands generally lead to the modulation of activity of the receptor. The term is intended to encompass naturally occurring compounds, synthetic compounds and/or recombinantly produced compounds. As used herein, this term can encompass agonists, antagonists, and inverse agonists.

As used herein, the terms “ligand-induced internalization,” “ligand-induced receptor internalization,” “ligand-induced receptor-mediated endocytosis” and the like are used interchangeably to refer to a process by which a ligand binds to a receptor on the surface of the cell membrane and the resulting ligand-receptor complex is internalized by the cell, i.e., moves into the cytoplasm of the cell (e.g., a cancer cell) or a compartment within the cytoplasm of the cell (endosomes, vesicles etc.) without causing irreparable damage to the cell membrane. Internalization may be followed up by dissociation of the resulting complex within the cytoplasm.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum. Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars), marine mammals (e.g., dolphins, whales), reptiles (e.g., snakes, frogs, lizards), and fish. In specific embodiments, the subject is a primate such as a human. However, it will be understood that the aforementioned terms do not imply that symptoms are present.

The term “receptor” as used herein refers to a protein normally found on the surface of a cell (e.g., EGFR) which, when activated, leads to a signaling cascade in the cell.

As used herein, in the context of a cancer, the term “responder” refers to a patient who exhibits or is more likely to exhibit a beneficial clinical response following treatment with an EGFR antagonist. By contrast, the term “non-responder,” as used herein, refers to a patient who is does not exhibit or is less likely to be exhibit a beneficial response following treatment with an EGFR antagonist. As used herein in the context of patient response to treatment with an EGFR antagonist, the terms “beneficial response,” “beneficial patient response,” and “clinically beneficial response,” “clinical benefit,” and the like, are used interchangeably and refer to favorable patient response to a drug as opposed to unfavorable responses, i.e., adverse events. In individual patients, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment. Evaluation of patients in assessing symptoms and/or severity of the disease may be carried out by various methods, which are known in the art. The evaluation may take into account numerous criteria, as determined by suitable biochemical, physiological, and/or behavioral factors.

As used herein, the terms “treatment,” “treating,” and the like, refer to administering an agent, or carrying out a procedure (e.g., radiation, a surgical procedure, etc.) to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a cancer) and/or adverse effect attributable to the disease or condition. These terms also cover any treatment of a condition or disease in a mammal, particularly in a human, and include: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; (c) relieving the disease, i.e., causing regression of the disease; (d) reducing the severity of a symptom of the disease and/or (e) reducing the frequency of a symptom of the disease or condition.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth and include As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, colorectal cancer, breast cancer, ovarian cancer, lung cancer, prostate cancer, hepatocellular cancer, gastric cancer, pancreatic cancer, cervical cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In an exemplary embodiment, the cancer is squamous cell carcinoma.

The term “tumor sample” as used herein means a sample comprising tumor material obtained from a cancerous patient. The term encompasses clinical samples, for example tissue obtained by surgical resection and tissue obtained by biopsy, such as for example a core biopsy or a fine needle biopsy. The term also encompasses samples comprising tumor cells obtained from sites other than the primary tumor, e.g., circulating tumor cells, as well as well as preserved tumor samples, such as formalin-fixed, paraffin-embedded tumor samples or frozen tumor samples. The term encompasses cells that are the progeny of the patient's tumor cells, e.g., cell culture samples derived from primary tumor cells or circulating tumor cells. The term encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

Each embodiment described herein is to be applied mutatis mutandis to each and every embodiment unless specifically stated otherwise.

### 2. Methods for Classifying EGFR Positive Tumors

The present invention provides methods for classifying tumors into EGFR antagonist sensitive and EGFR antagonist resistant subtypes. In accordance with the present invention, these methods involve analyzing the ligand-induced EGFR internalization status of the tumor. A detected ligand-induced EGFR internalization that is impaired or abrogated relative to a control (e.g., a normal EGFR-expressing cell) classifies the tumor as EGFR antagonist sensitive, whereas a detected ligand-induced EGFR internalization that is the same as, similar to, or even greater than the control classifies the tumor as EGFR antagonist resistant. In some embodiments, impaired ligand-induced EGFR internalization is indicated when, suitably after at least 10 minutes (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more minutes) in the presence of an EGRF ligand (e.g., EGF, TGF-α, amphiregulin, heparin-binding EGF-like growth factor, betacellulin, and epiregulin): (a) at least 90% (e.g., 90%, 91%, 92%, 93%, 94%, 95%, 96%, 97%, 98%, 99% or 100%) of the EGFR in EGFR-expressing cells of the tumor is localized or remains localized to the plasma membrane (e.g., basolateral membrane) of the cells; (b) the ratio of EGFR localized to the plasma membrane of EGFR-expressing cells of the tumor to EGFR localized in the intracellular compartments of those cells (e.g., cytoplasm, nucleus etc.) is selected from 90:10, 91:9, 92:8, 93:7, 94:6, 95:5, 96:4, 97:3, 98:2, 99:1 or 100:0; or (c) the degree or quantum of ligand-induced EGFR internalization in EGFR-expressing cells of the tumor is less than 10%, 9%, 8%, 7%, 6%, 5%, 4%, 3%, 2%, 1% of the degree or quantum of ligand-induced EGFR internalization in EGFR-expressing normal cells.

In some embodiments, unimpaired ligand-induced EGFR internalization is indicated when, suitably after at least 10 minutes (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more minutes) in the presence of an EGRF ligand (e.g., EGF, TGF-α, amphiregulin, heparin-binding EGF-like growth factor, betacellulin, and epiregulin): (a) less than 100% (e.g., 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 5% or even less) of the EGFR in EGFR-expressing cells of the tumor is localized or remains localized to the plasma membrane (e.g., basolateral membrane) of the cells; (b) the ratio of EGFR localized to the plasma membrane of EGFR-expressing cells of the tumor to EGFR localized in the intracellular compartments of those cells (e.g., cytoplasm, nucleus etc.) is selected from 99:1; 98:2, 97:3, 96:4, 95:5, 94:6, 93:7, 92:8, 91:9, 90:10, 85:15, 80:20, 75:25, 70:30, 65:35, 60:40, 55:45, 50:50, 45:55, 40:60, 35:65; 30:70, 25:75, 20:80, 15:85, 10:90, 5:95 or 0:100; (c) the degree or quantum of ligand-induced EGFR internalization in EGFR-expressing cells of the tumor varies by less than 10%, 9%, 8%, 7%, 6%, 5%, 4%, 3%, 2% or 1% as compared to the degree or quantum of ligand-induced EGFR, internalization in EGFR-expressing normal cells; or (d) the degree or quantum of ligand-induced EGFR internalization in EGFR-expressing cells of the tumor is greater than the degree or quantum of ligand-induced EGFR internalization in EGFR-expressing normal cells.

In some embodiments, the methods comprise: (a) providing a tumor sample comprising one or a plurality of EGFR-expressing tumor cells or putative EGFR-expressing tumor cells, (b) contacting the tumor cell(s) with a labeled EGFR ligand to form a labeled complex comprising EGFR and the labeled EGFR ligand, and (c) monitoring ligand-induced EGFR internalization in the tumor cells by detecting cellular location of the labeled complex. Typically, the methods further comprise determining the degree of ligand-induced EGFR internalization by comparing the amount of labeled complex bound to the surface of the tumor cells and the amount of labeled complex inside the tumor cells (e.g., intracellular compartment of the tumor cells including, but not limited to, cytoplasm, nucleus, endosomes, etc.). In some embodiments, ligand-induced EGFR internalization is detected by qualitatively or quantitatively detecting a decrease of labeled complex on the surface of the tumor cells and/or qualitatively or quantitatively detecting an increase of labeled complex inside the tumor cells. Usually, ligand-induced EGFR internalization in the tumor cells is monitored for at least 10 and less than 60 minutes, usually for at least 20 and less than 40 minutes.

Generally, the methods further comprise providing a control sample comprising one or a plurality of EGFR-expressing control cells (e.g., normal cells), (b) contacting the control cell(s) with a labeled EGFR ligand, which is generally the same as the one used for contacting the tumor cells, to form a labeled complex comprising EGFR and the labeled EGFR ligand, and (c) monitoring ligand-induced EGFR internalization in the control cells by detecting cellular location of the labeled complex. Typically, these methods further comprise determining the degree of ligand-induced EGFR internalization by comparing the amount of labeled complex bound to the surface of the control cells and the amount of labeled complex inside the control cells (e.g., intracellular compartment of the tumor cells including, but not limited to, cytoplasm, nucleus, endosomes, etc.). Usually, ligand-induced EGFR internalization in the control cells is monitored for the same time employed for the tumor cells.

In some embodiments, the degree or amount of ligand-induced EGFR internalization in the control cells is compared with the degree or amount of ligand-induced EGFR internalization in the tumor cells to determine whether the tumor cells have impaired or unimpaired ligand-induced EGFR internalization. In illustrative examples of this type, impaired ligand-induced EGFR internalization in the tumor cells is determined when the ligand-induced EGFR internalization in the tumor cells is less than 10%, 9%, 8%, 7%, 6%, 5%, 4%, 3%, 2%, 1% of the degree or quantum of ligand-induced EGFR internalization in EGFR-expressing normal cells. In other illustrative examples, unimpaired ligand-induced EGFR internalization in the tumor cells is determined when the degree or quantum of ligand-induced EGFR internalization in EGFR-expressing cells of the tumor varies by less than 10%, 9%, 8%, 7%, 6%, 5%, 4%, 3%, 2% or 1% as compared to the degree or quantum of ligand-induced EGFR internalization in EGFR-expressing normal cells; or the degree or quantum of ligand-induced EGFR internalization in EGFR-expressing cells of the tumor is greater than the degree or quantum of ligand-induced EGFR internalization in EGFR-expressing normal cells.

In some embodiments, the methods further comprise obtaining the tumor sample from a subject with a cancer, suitably an EGFR positive cancer. The sample may, for example, be a fresh biopsy sample, a fixed sample, e.g. a formalin fixed, paraffin-embedded (FFPE) sample, or a frozen sample. Non-limiting examples of EGFR positive cancers include squamous cell carcinoma (e.g., epithelial squamous cell carcinoma), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. The tumor may be a metastatic or non-metastatic tumor.

The methods further comprise classifying the tumor as an EGFR antagonist sensitive tumor if the detected ligand-induced EGFR internalization in the tumor cells is impaired or abrogated relative to the control cells, or classifying the tumor as an EGFR antagonist resistant tumor if the detected ligand-induced EGFR internalization in the tumor cells is the same as, similar to, or even greater than the control cells.

Ligand-induced EGFR internalization may be carried out by any suitable means. Receptor internalization assays are well known in the art, representative examples of which are described in Fukunaga et al. (2006) Life Sciences 80(1):17-23; Bernhagen et al. (2007) Nature Medicine 13:587-596; natureprotocols.com/2007/04/18/receptor_internalization_assay.php). One well-known method to determine receptor internalization is to tag a ligand with a fluorescent label, e.g., a fluorescers or fluorescent dye such as Alexa Fluor 488, fluorescein isothiocyanate, Texas red, rhodamine, and the like, a fluorescent protein such as Green Fluorescent Protein (GFP), or other suitable labeling agent. Upon binding of the ligand to the receptor, fluorescence microscopy may be used to visualize receptor internalization. Similarly, EGFR may be tagged with a labeling agent and fluorescence microscopy may be used to visualize receptor internalization. If ligand-induced EGFR internalization is reduced in tested cells, lessened internalization of fluorescence will be observed in those cells as compared to appropriate control cells (e.g., fluorescence may be observed only at the periphery of the cell where EGFR ligand binds EGFR rather than in endosomes or vesicles). Of course, other labels may be employed instead of fluorescent labels, including: chemiluminescent compounds such as luciferin; 2,3-dihydrophthalazinediones such as luminol; radioactive labels such as ³H, ¹²⁵I, ³⁵S, ¹⁴C, or ³²P; enzymes such as horse radish peroxidase, alkaline phosphatase and others commonly used in immunoassays, and colorimetric labels such as colloidal gold or colored glass or plastic beads such as polystyrene, polypropylene or latex.

In some embodiments, receptor internalization assays may involve the detection or quantification of EGFR using immunological binding assays (e.g., when using a radiolabeled antibody to detect the amount of EGFR ligand or EGFR on the cell surface during a receptor internalization assay). Immunological binding assays are widely described in the art (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Ten, eds., 7th ed. 1991). Commonly used immunoassays include noncompetitive assays, e.g., sandwich assays, and competitive assays.

In advantageous embodiments, the receptor internalization assays used for the tumor classification methods of the present invention are those described in the examples.

### 3. Methods for Stratifying Responders and Non-Responders and Uses Therefor

The tumor classification methods of the present invention are useful for stratifying cancer-affected subjects into EGFR antagonist responders and EGFR antagonist non-responders. Thus, when a subject's tumor is determined as having an impaired or abrogated ligand-induced EGFR internalization status, the subject is stratified as a responder to EGFR antagonist therapy. Conversely, when a subject's tumor is determined as having an unimpaired ligand-induced EGFR internalization status, the subject is stratified a non-responder to EGFR antagonist therapy. This stratification, in turn permits, better management of cancer-affected subjects in which responders are administered an EGFR antagonist and non-responders are offered alternate treatment.

Thus, subjects identified as responders are administered EGFR antagonists in effective amounts to treat the cancer. Representative EGFR antagonists include compounds that bind to or otherwise interact directly with EGFR and prevent or reduce its signaling activity. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX™) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see, WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-α for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al. (2004) J. Biol. Chem. 279(29):30375-30384). The anti-EGFR antibody may be conjugated with a cytotoxic agent, thus generating an immunoconjugate (see, e.g., EP659,439A2, Merck Patent GmbH). In some embodiments, the EGFR antagonists are small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA™ Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quina-zolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoli-ne, AstraZeneca); ZM 105180 ((6-amino-4-(3-methylphenyl-amino)-quinazoline, Zeneca); BIBX-1382 (N-8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Sugen); and AG1571 (SU 5271; Sugen).

Conversely, subjects identified as non-responders are administered are administered an alternative therapy to treat the cancer, non-limiting examples of which include radiotherapy, surgery, chemotherapy, hormone ablation therapy, pro-apoptosis therapy and immunotherapy.

3.1 Radiotherapy

Radiotherapies include radiation and waves that induce DNA damage for example, γ-irradiation, X-rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

3.2 Chemotherapy

Chemotherapeutic agents may be selected from any one or more of the following categories:

(i) antiproliferative/antineoplastic drugs and combinations thereof, as used in medical oncology, such as alkylating agents (e.g., cis-platin, carboplatin, cyclophosphamide, nitrogen mustard, melphalan, chlorambucil, busulphan and nitrosoureas); antimetabolites (e.g., antifolates such as fluoropyridines like 5-fluorouracil and tegafur, raltitrexed, methotrexate, cytosine arabinoside and hydroxyurea; anti-tumor antibiotics (e.g., anthracyclines like adriamycin, bleomycin, doxorubicin, daunomycin, epirubicin, idarubicin, mitomycin-C, dactinomycin and mithramycin); antimitotic agents (e.g., vinca alkaloids like vincristine, vinblastine, vindesine and vinorelbine and taxoids like paclitaxel and docetaxel; and topoisomerase inhibitors (e.g., epipodophyllotoxins like etoposide and teniposide, amsacrine, topotecan and camptothecin);

(ii) cytostatic agents such as antioestrogens (e.g., tamoxifen, toremifene, raloxifene, droloxifene and iodoxyfene), estrogen receptor down regulators (for example fulvestrant), antiandrogens (e.g., bicalutamide, flutamide, nilutamide and cyproterone acetate), UH antagonists or LHRH agonists (e.g., goserelin, leuprorelin and buserelin), progestogens (e.g., megestrol acetate), aromatase inhibitors (e.g., as anastrozole, letrozole, vorazole and exemestarie) and inhibitors of 5α-reductase such as finasteride;

(iii) agents which inhibit cancer cell invasion (e.g., metalloproteinase inhibitors like marimastat and inhibitors of urokinase plasminogen activator receptor function);

(iv) inhibitors of growth factor function, for example such inhibitors include growth factor antibodies, growth factor receptor antibodies (e.g., the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;

(v) anti-angiogenic agents such as those which inhibit the effects of vascular endothelial growth factor, (e.g., the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (e.g., linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense; and

(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

3.3 Immunotherapy

Immunotherapy approaches, include for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

3.4 Other Therapies

Examples of other cancer therapies include phototherapy, cryotherapy, toxin therapy or pro-apoptosis therapy. One of skill in the art would know that this list is not exhaustive of the types of treatment modalities available for cancer and other hyperplastic lesions.

Generally, the therapeutic agents described above are administered in the form of pharmaceutical compositions that optionally comprise a pharmaceutically acceptable carrier, excipient and/or stabilizer (Remington's Pharmaceutical Sciences 16th edition, Osol, A. Ed. (1980)). These compositions are generally in the form of lyophilized formulations or aqueous solutions. Antibody crystals are also contemplated (see, U.S. Pat. Appl. 2002/0136719). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized antibody formulations are described in WO 97/04801.

Exemplary anti-EGFR antibody formulations include pertuzumab formulations for therapeutic use, which generally comprise 30 mg/mL pertuzumab in 20 mM histidine acetate, 120 mM sucrose, 0.02% polysorbate 20, at pH 6.0. An alternate pertuzumab formulation comprises 25 mg/mL pertuzumab, 10 mM histidine-HCl buffer, 240 mM sucrose, 0.02% polysorbate 20, pH 6.0. Cetuximab (ERBITUX™) formulations are commercially available as a sterile liquid formulation intended for intravenous infusion. A typical formulation contains (per vial) 100 mg cetuximab, 424 mg sodium chloride, 20 mg sodium dihydrogen phosphate dihydrate, 66 mg disodium phosphate dihydrate and water for injection ad 50 mL.

The pharmaceutical compositions may also contain more than one active compound as necessary for the particular indication being treated, desirably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

### 4. Kits

As noted above, the present invention contemplates the use of receptor internalization assays for determining the ligand-induced EGFR internalization status of a tumor, and/or the EGFR antagonist sensitivity of a tumor, and/or stratifying a subject into a treatment subgroup selected from EGFR antagonist responder and non-responder. All the essential materials and reagents (e.g., labels, antibodies, ligands etc.) required for these assays may be assembled together in a kit. The kits may also optionally include appropriate reagents for detection of labels, positive and negative controls, washing solutions, blotting membranes, microtiter plates dilution buffers and the like. Such kits also generally will comprise, in suitable means, distinct containers for each individual reagent. The kit can also feature various devices and reagents for performing one of the assays described herein; and/or printed instructions for using the kit for assaying receptor internalization.

In some embodiments, the methods described generally herein are performed, at least in part, by a processing system, such as a suitably programmed computer system. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to assay receptor internalization. A processing system also can be used to determine the ligand-induced receptor internalization status of a tumor, and to stratify a subject into a treatment subgroup selected from therapeutic antibody responders and non-responders, on the basis of the receptor internalization status. In some examples, commands inputted to the processing system by a user assist the processing system in making these determinations.

In one example, a processing system includes at least one microprocessor, a memory, an input/output device, such as a keyboard and/or display, and an external interface, interconnected via a bus. The external interface can be utilized for connecting the processing system to peripheral devices, such as a communications network, database, or storage devices. The microprocessor can execute instructions in the form of applications software stored in the memory to allow a process (e.g., determination of ligand-induced EGFR internalization status, and/or determination of EGFR antagonist sensitivity of a tumor, and/or stratification of a subject into a treatment subgroup selected from EGFR antagonist responder and non-responder) to be performed, as well as to perform any other required processes, such as communicating with the computer systems. The applications software may include one or more software modules, and may be executed in a suitable execution environment, such as an operating system environment, or the like.

In order that the invention may be readily understood and put into practical effect, particular preferred embodiments will now be described by way of the following non-limiting examples.

### EXAMPLES

### Example 1

### Disregulation of EGFR in Keratinocyte Tumors

### Materials and Methods

Cell lines include seven lines from HNSCC including examples derived from the tongue, pharynx and hypopharynx; SCC-9, SCC-15, SCC-25, Detroit-562, Cal 27, FaDu, and Colo-16. In addition a further two lines have been used, KJD, derived from transformed human epidermal keratinocytes and A-431, derived from, a vulval SCC and known to overexpress EGFR. Cell line identities were verified by SNP analysis. Cells were mycoplasma free and tested regularly. Cells were grown in Ham's F-12 media: DMEM supplemented with 10% FBS, 10 mM HEPES, 2 mM L-Glutamine, 1 mM Sodium pyruvate (Gibco, Invitrogen). For basal conditions cells were grown for 3 hrs in complete media but without 10%. FBS. Other reagents included Alexa488-labelled EGF (Invitrogen), EGFR (clone 31G7; Invitrogen) Alexa-594 goat anti-mouse IgG (A11005; Invitrogen), HRP-conjugated goat anti-mouse IgG (A10547; Invitrogen), Mouse anti-flotillin-1 IgG (610821; BD Transduction Laboratories).

Sample Acquisition

Patients with suspected SCC or intra-epidermal carcinoma (IEC) are identified by doctors. Live tissue samples are transported on ice to the department of anatomical pathology, where a trained pathologist dissects the sample and provides a suitable portion of tissue for laboratory investigation. Whilst actinic keratoses are not specifically sought for this study as they would not usually be excised, any biopsied lesions which subsequently prove to be actinic keratosis (AK) may be included and uninvolved skin tissue is acquired from the margins of excision specimens.

EGFR Stimulation and Uptake Studies

In brief, the technique used for sample preparation is as follows. Tissue samples are collected from clinical areas immediately after excision from the patient. A small, representative section is provided by the pathologist and this is sectioned in a particular manner. Samples are oriented in a petri dish such that they can be cut vertically producing section approximately 1 mm in thickness and encompassing the full thickness of the specimen less any subcutaneous fat which is excised prior to sectioning. Sectioning is performed by hand using a scalpel or razor blade. Samples are then treated in cold serum-free media washes for up to one hour. EGF (EGF-Alexa Fluor 488-Streptavidin—Invitrogen) is added to the SFM and the samples incubated at 37° C. for various time points. Some samples do not have EGF added as a control and others first have Cetuximab added and are incubated for 30 minutes prior to the addition of EGF as a control in which ligand binding should be blocked. After incubation for the appropriate time points, uptake is stopped by cooling in ice and washing in cold PBS with 0.1% Triton X-100 (PBTx). After several washes samples are fixed in 4% paraformaldehyde overnight.

Whole Mount Immunofluorescence

Samples are subjected to a bleaching protocol using Dent's bleach followed by a methanol series to bring the samples back to aqueous. Immunofluorescence can then be performed using various antibodies with excess binding blocked by incubation in 10% Horse serum in PBTx. In most cases anti-EGFR antibody (Mouse anti-EGFR, clone 31 G7—Invitrogen) is used to visualize the receptors, however in the case of large tumor samples with multiple sections available, other antibodies may be used additionally such as anti-EEA1 and anti-clathrin antibodies. After this an appropriate secondary antibody conjugated to Alexa is used and DAPI is added for nuclear staining. Some samples are exposed to secondary antibody only serving as a staining control. Samples are then mounted on slides with a central depression using ProLong Gold™ anti-fade mounting reagent (Invitrogen) and sealed prior to imaging with the Zeiss confocal microscope.

Imaging

Slides were imaged using a Zeiss 510 Meta laser confocal microscope with a 63× objective with Zen 2008 software. Slides were examined for histological features to confirm localization to the epidermis at both 63× and 25×. Features noted were the presence of the dermo-epidermal junction and basement membrane, typical appearance of the dermis with autofluorescent collagen and paucity of cells, a typical appearance of the epidermis with stratified squamous epithelium and in dysplastic tissue, irregularity with nuclear pleomorphism. The thickness of the epidermis is significant as epidermal thickening and hyperplasia is a feature of dysplastic lesions. In the stratum corneum also, thickening can be seen as well as parakeratosis, both features of dysplastic lesions. Multiple regions of epidermis or nests of invasive keratinocytes are imaged at 63× to clearly show the distribution of EGF488. Where possible z-stack images are taken of representative tumor regions. Images are taken at 25× magnification or tile scans at 63× to show overall features of the specimen.

Image Analysis

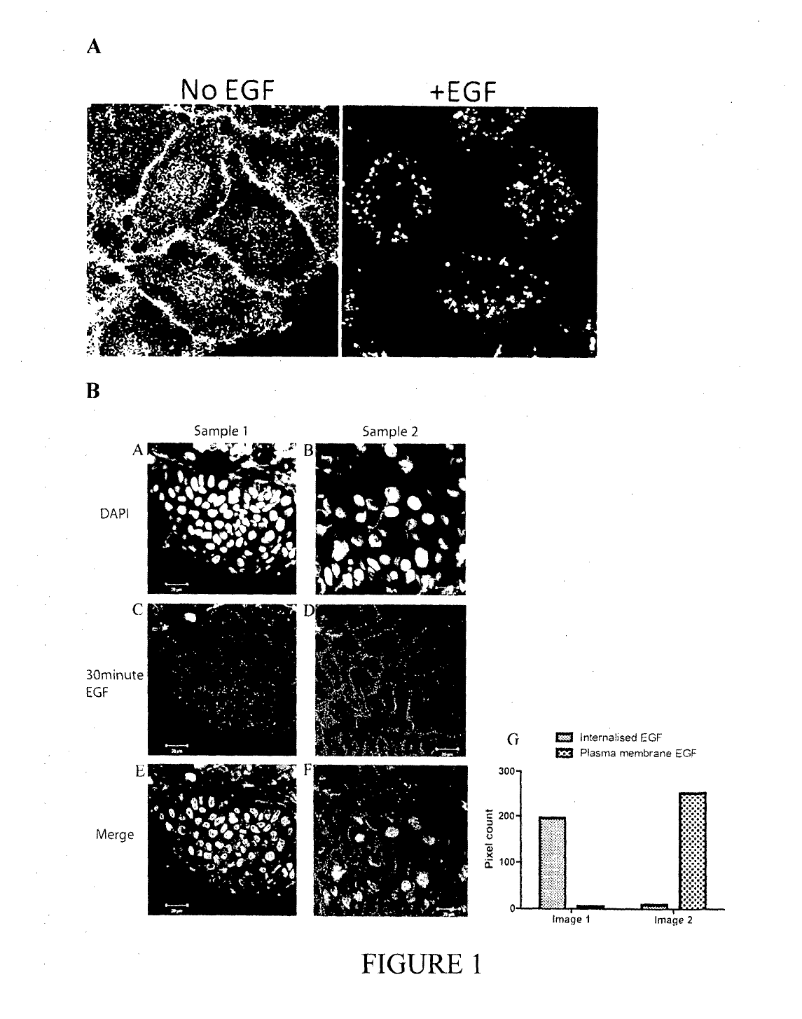

Images were reviewed with an experienced researcher in the use of cellular immunofluorescence techniques (supervisor FS) and categorized on the basis of the principle appearance of the distribution of Alexa-EGF488 as either ‘internalized’ or ‘disregulated’. Internalized tumor samples predominantly show a punctate staining pattern corresponding to internalized receptors grouped into endosomal structures at varying distances from the nucleus. After 30 minutes of stimulation with EGF-488, disregulated examples show punctate or semi-confluent staining in a distribution consistent with the location of the plasma membrane (FIG. 1).

Quantification of the degree of uptake was performed using Photoshop software. This process generated a data output which can be graphically represented to show degree of internalized versus disregulated for each tumor sample based on as many images as are available.

Nuclear morphometry analysis was performed using Image J software. Confocal images taken at 63× with a size of 142.6×142.6 micrometers were utilized. Tumor images were taken from representative portions of the tumor showing EGF-488 or EGFR positivity. A standard size area was taken from each image of 2500 μm², usually in the form of a 50×50 μm². The region sampled included a portion of the basal cell layer, where the highest level of EGF uptake was noted, wherever possible. Areas excluded regions of dermis or other areas of low cellularity and those with high levels of abnormal or non specific appearing coloration such as caused by autofluoresence. Images were thresholded to highlight as much of all the nuclei as possible and then converted to binary. The watershed tool was used to insert divisions between nuclei. The resultant image was checked for similarity with the original image. Adequate binary images were then analyzed automatically. This resulted in numbering of all nuclei included and generation of morphometric data set. These included; nuclear Cross-sectional area, Perimeter, Best fitting ellipse data (length of major and minor diameters and angle of major axis from the x axis of the image), Feret data (Longest caliper/feret distance,) and shape parameters principally Aspect ratio (length of major diameter of best fitting ellipse divided by length of minor diameter). Data generated from this analysis was plotted and analyzed using Graphpad Prism™ 5′ software package. Comparisons between tumor tissue and adjacent skin controls and between different tumors were performed using Mann-Whitney U test for unpaired data.

EGFR expression was assessed digitally on images of tumor and control tissue after immunofluorescence with a specific antibody. Software package NIS-elements™ (Nikon) was used with settings standardized against an example of a highly EGFR expressing SCC and a very low expressing control skin sample.

### Results

Tumor Analysis and EGF Uptake

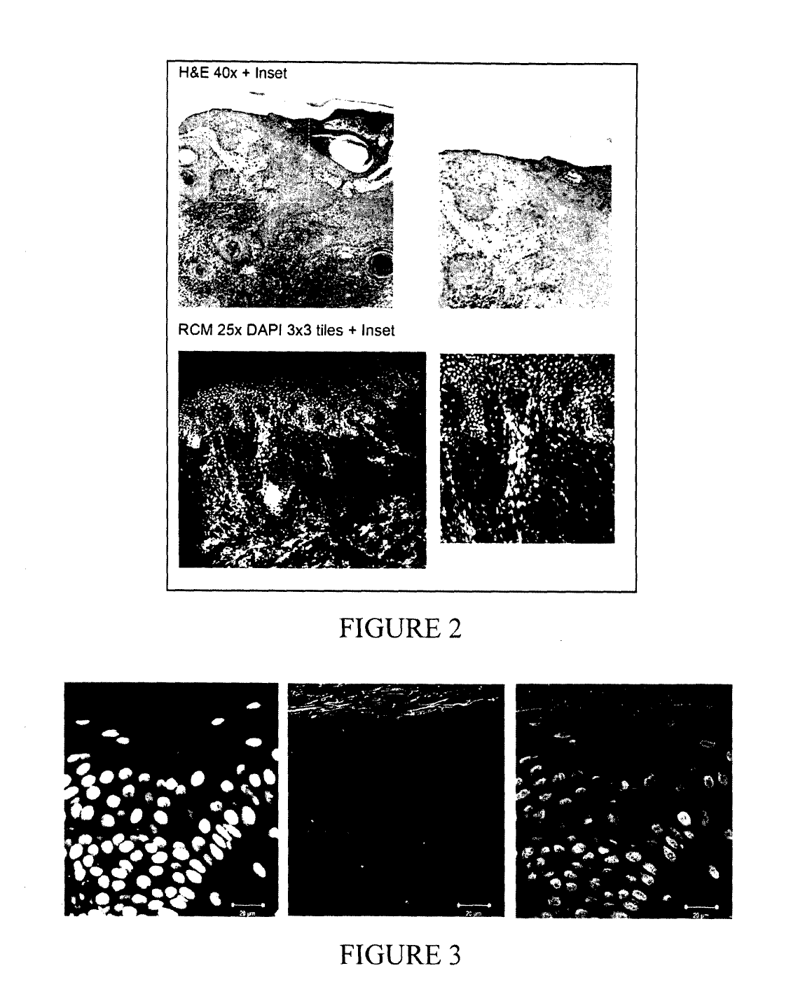

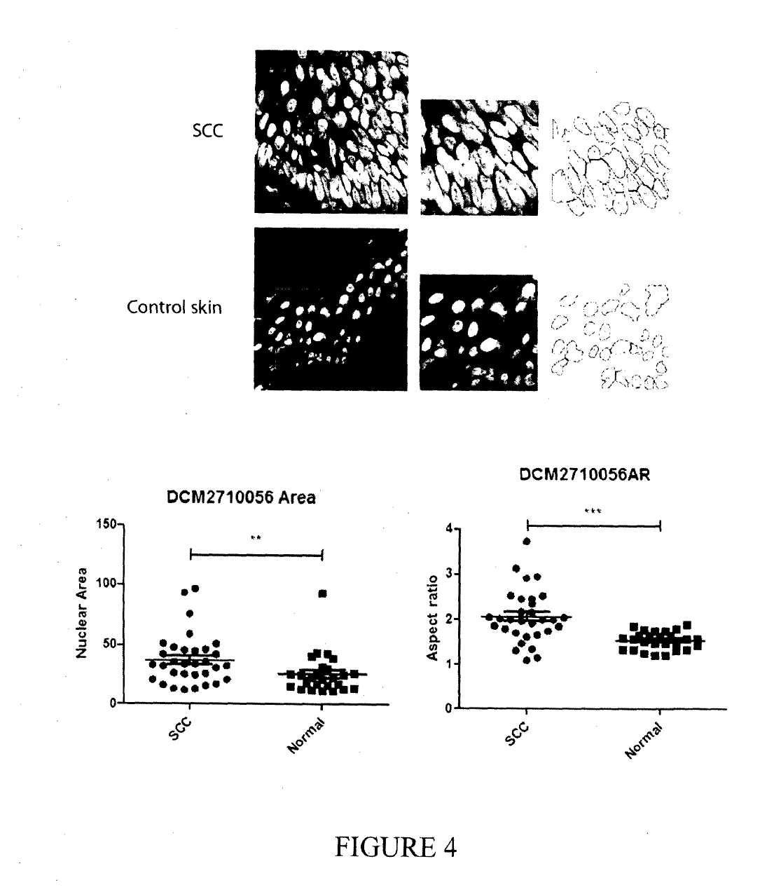

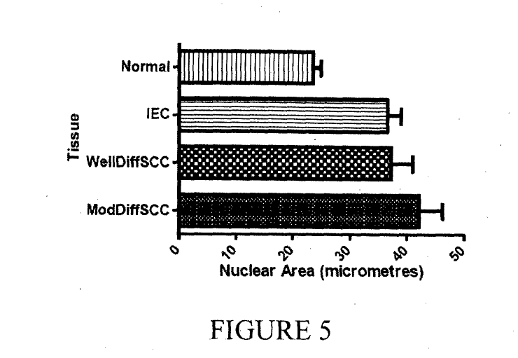

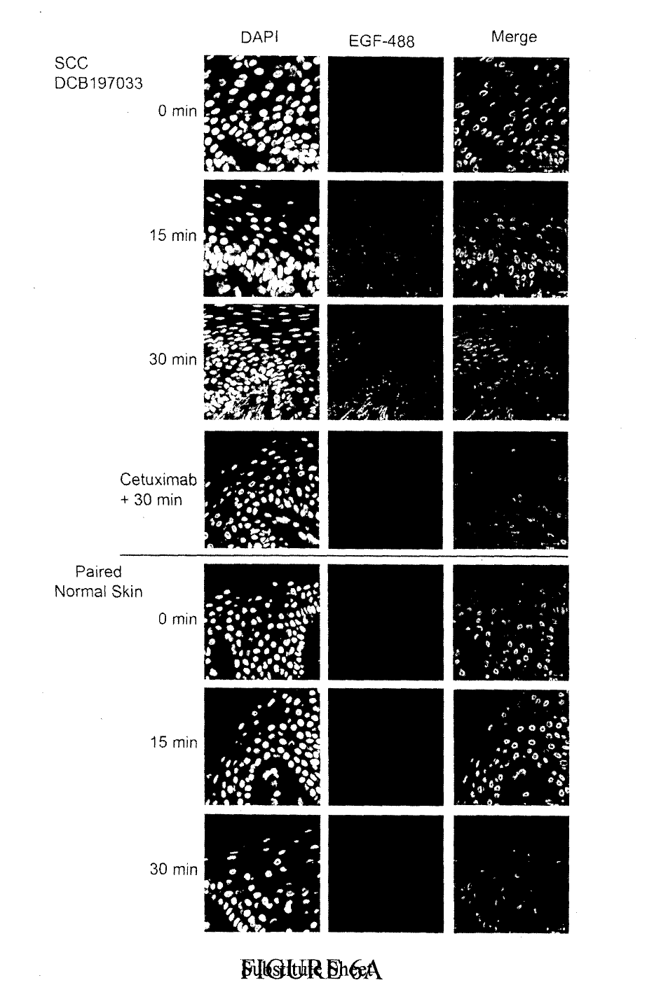

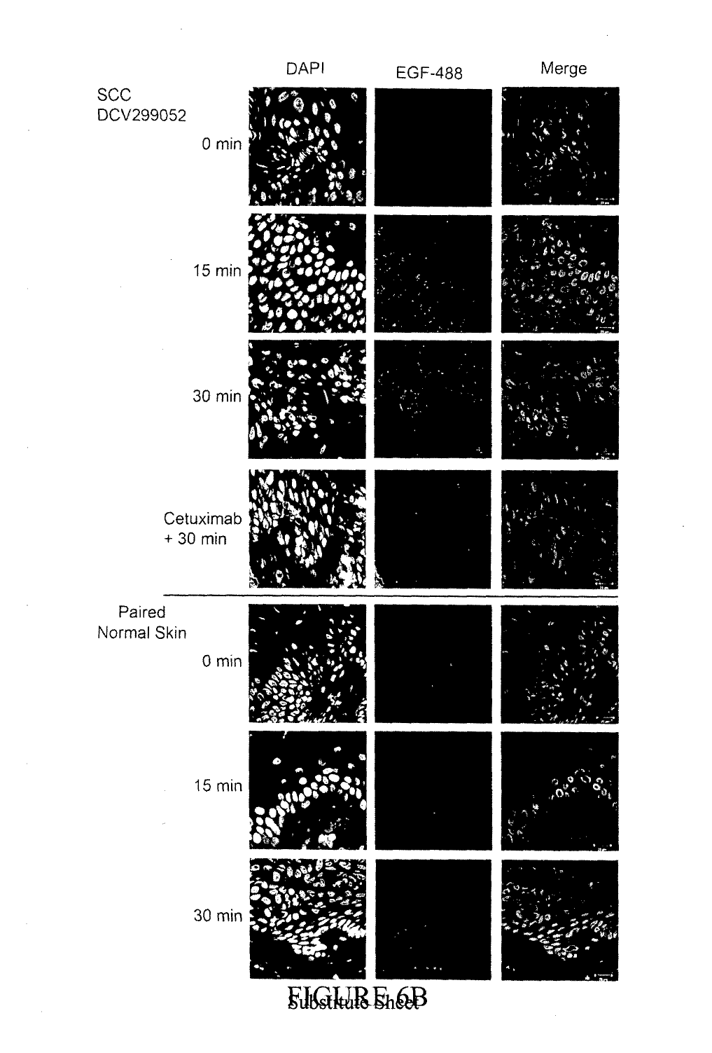

Optimization of cutaneous sample analysis by whole mount confocal imaging was undertaken. Careful sectioning by hand was sufficient to demonstrate all vertical regions of the skin on processed and mounted samples. This allowed each section to be assessed for a limited array of histological features of the corresponding pathological diagnosis (FIGS. 2 and 3). In addition to this, initial analysis of nuclear morphometry demonstrated significant differences between tumor tissue and control skin (FIG. 4) and differences, although not significant in nuclear area between different pathological types of squamous lesions (FIG. 5).

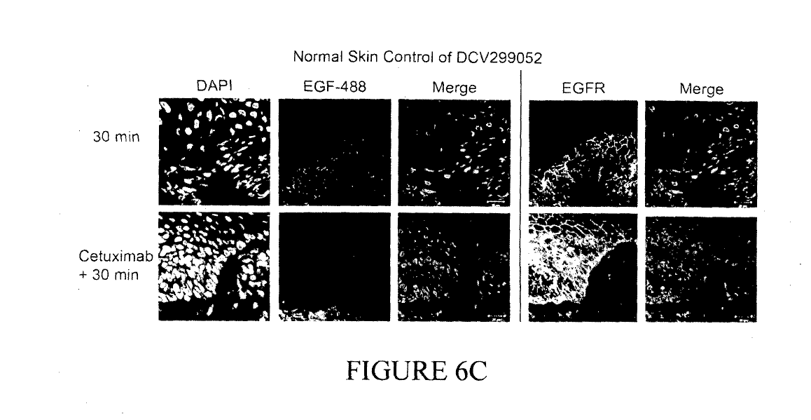

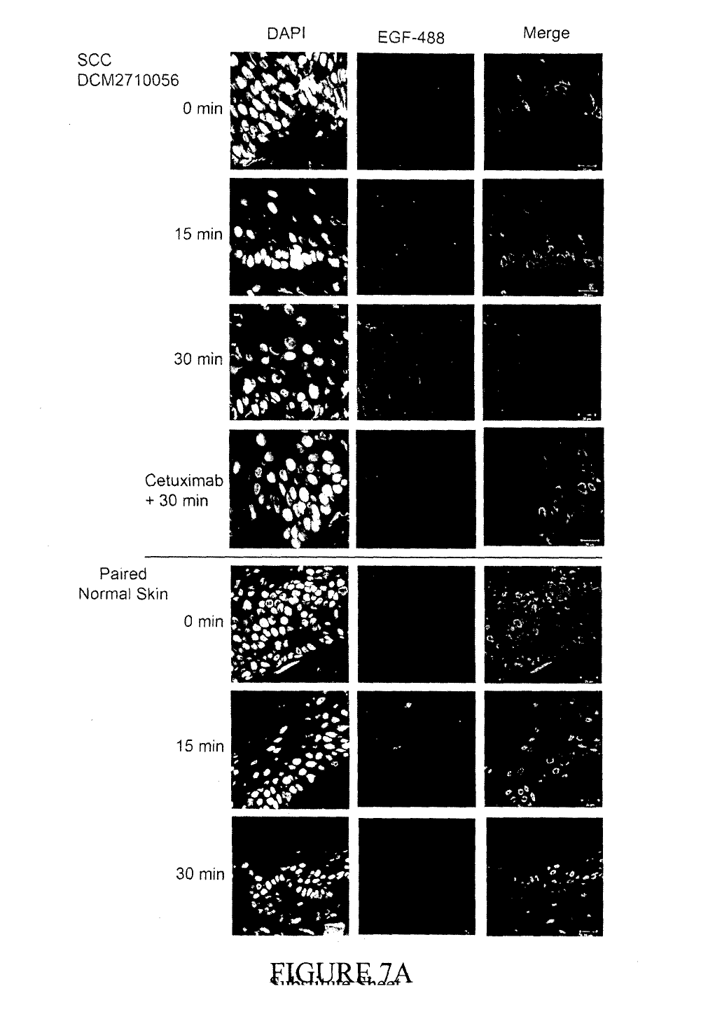

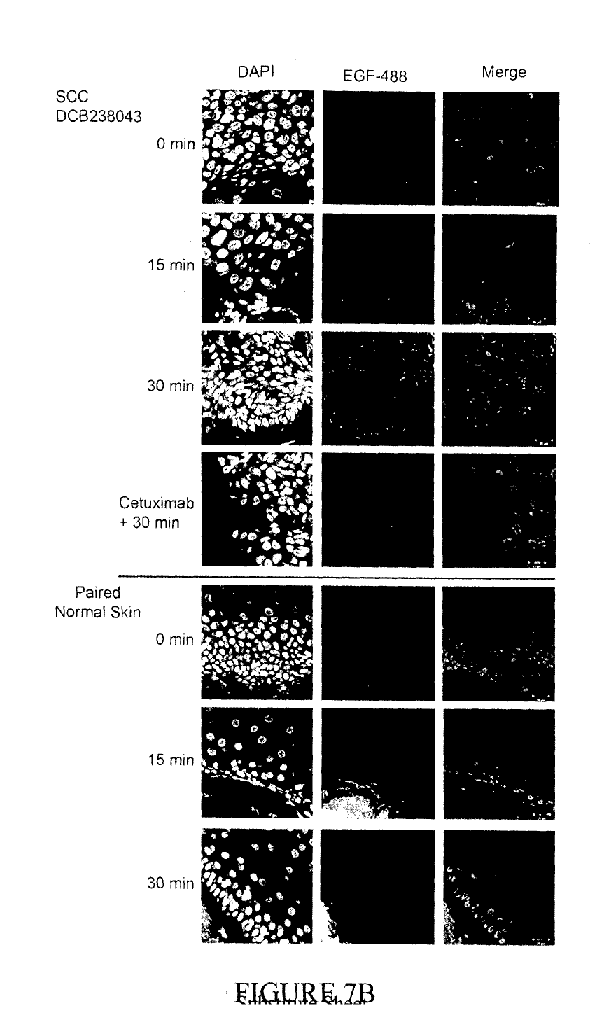

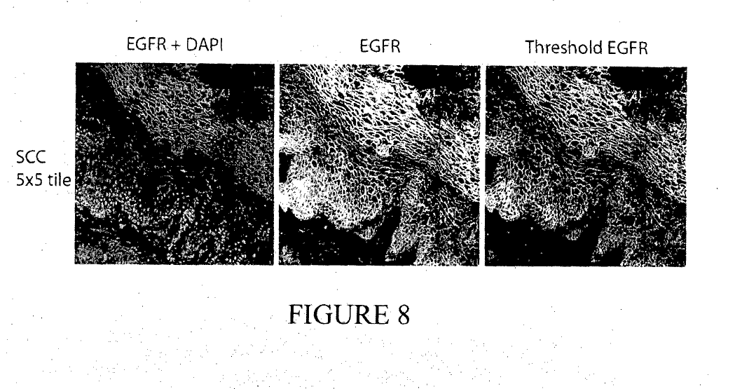

For each sample EGF uptake studies were performed on the fresh tumor specimens using the methods described. Control skin samples if available were also subjected to the protocol. Sample images were reviewed with a supervisor (FS) and a determination made as to the predominant localization of the labeled EGF at the 30-minute time point (FIG. 6). In a small number of cases adequate 30 minute samples were not available and the determination was made on a 15 minute time point sample. In the majority of cases with an adequate 30 minute time point result a determination could be made at the 15 minute time point which corresponded to the 30 minute result. As a result, tumors were categorized as either ‘internalized’ if EGF is appreciably endocytosed on examination (FIG. 7) or ‘disregulated’ if the EGF appeared to be predominantly accumulated on the plasma membrane (FIG. 8).

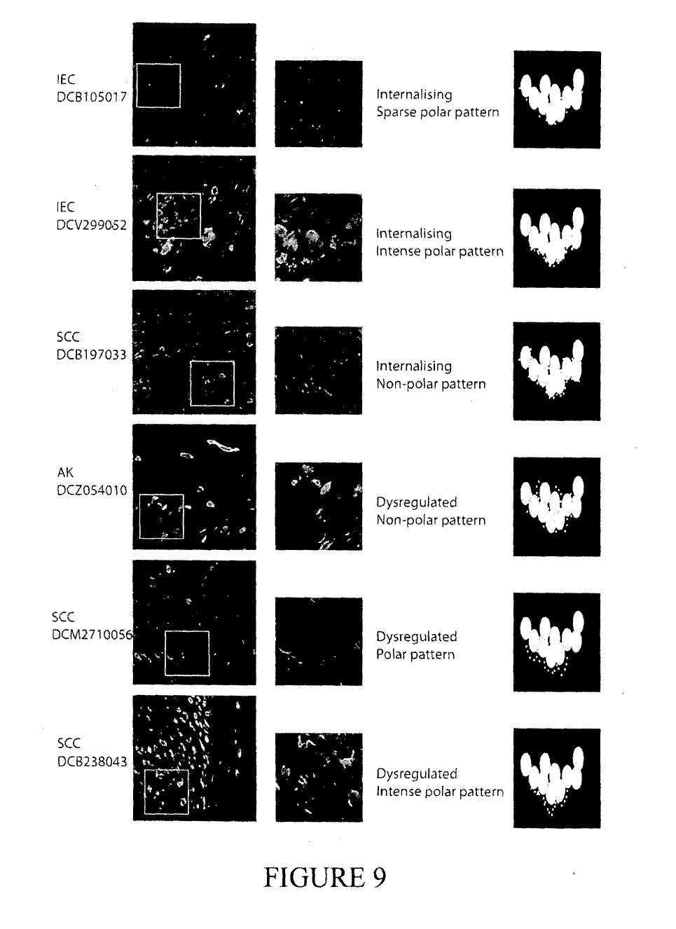

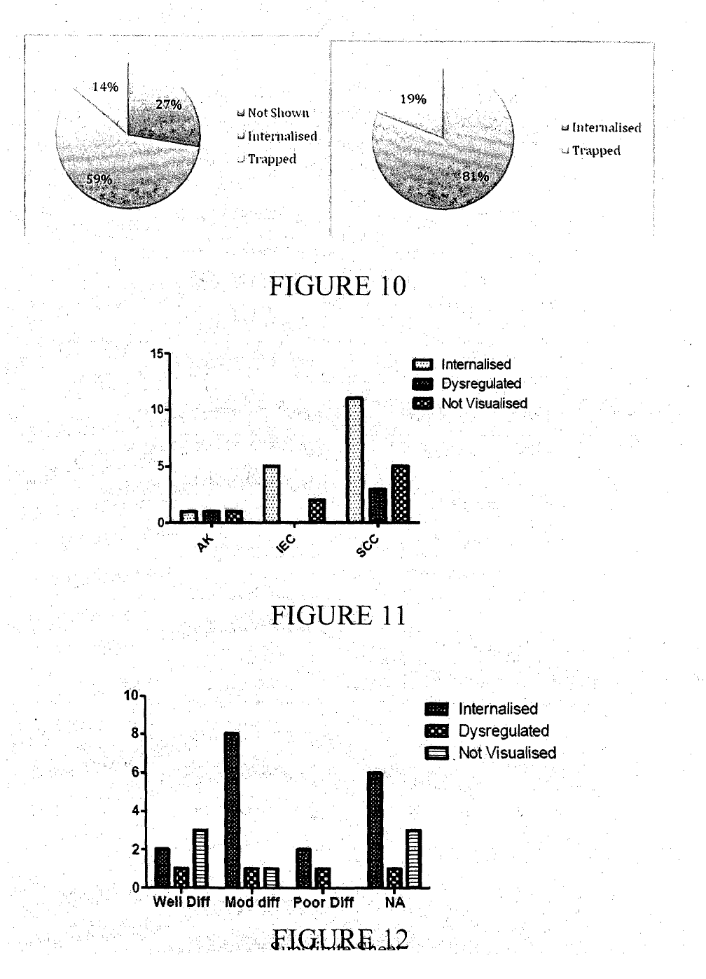

As expected, EGFR expression was most intense in the basal layers and at the invasive edges of tumors (FIG. 9). It was in this region that EGF-488 was usually visualized. Several different subtypes of EGF localization were noted in the most basal keratinocyte layers (FIG. 10). In particular, several tumors demonstrated marked basal polarization of EGF uptake. This is consistent with the invasive nature or potential of these lesions.

There were 26 patients who provided 29 lesions for examination. Patients were all of fair skin types I-III. Basic demographic information was collected along with significant data from the medical histories. Risk factors for the development of SCC and for advanced, recurrent or metastatic SCC were recorded wherever data was available. Important information regarding the patients' skin cancer history was also recorded. All patients had had a previous skin malignancy but not all had a previous SCC. Between 0-4 previous SCCs was most common with 11 patients having had >5 previous SCCs with a maximum of 18. A history of a previous high risk SCC was defined as a lesion which demonstrated any of the tumor-specific high risk factors outlined in Table 1.

TABLE 1
RISK FACTORS FOR RECURRENCE, METASTASES OR
DEATH IN CUTANEOUS SQUAMOUS CELL CARCINOMA
(CRANMER ET AL., 2010; WOLLINA, 2012)

| | |
|---|---|
| Gross/tumor | Lesion diameter >2 cm |
| factors | Indistinct margins on clinical examination |
| | Location at embryonic fusion planes or lips, ear |
| | or anogenital region |
| | Associated with burn, chronic wound, ulcer or |
| | irradiated skin |
| | Recurrent tumor |
| Histopathology | Depth >2 mm or Clark level IV (to reticular/deep |
| | dermis) |
| | Poorly differentiated or infiltrating histology |
| | Basosquamous or desmoplastic histology |
| | Perineural or lymphovascular invasion |
| | Positive surgical margins after resection of |
| | primary tumor |
| Host factors | Immunosuppression including organ transplantation |
| | Chronic Lymphocytic Leukemia |
| | Chronic dermatoses including; epidermolysis |
| | bullosa, xeroderma pigmentosum, epidermodysplasia |
| | verruciformis, albinism, pansclerotic morphea, |
| | hidradenitis suppurativa |

Sample characteristics are summarized in Table 2.

TABLE 2
PATIENT CHARACTERISTICS

| | |
|---|---|
| Age | Age mean 73.8 years (range 46-87) |
| Gender | M:F = 18:8 |
| High risk host | Concurrently or previously immunosuppressed |
| features | (n = 4, Indications-Vasculitis, Rheumatoid |
| | Arthritis, Nephrotic Syndrome, Lymphoma) |
| | CLL (n == 1) |
| Other SCC features | Number of previous SCC |
| in history | History of ≧1 previous high risk SCC |

Results of Tumor EGFR Trafficking Analysis

Tumors were characterized according to the grade or invasion stage noted on pathology and the EGF uptake status as outlined above. In eight cases (27%) EGF-488 was not seen and these could not be determined to be either internalizing or disregulated. There are a number of possibilities for this, including that the tissue was not viable by the time it reached the lab and uptake studies were performed. Of the remaining cases, 17 (59%) were seen to be EGF internalizing while 4 (14%) were disregulated (FIG. 11). A summary of tumor type and patient risk factors and trafficking status is shown in Table 3.

TABLE 3
TUMOR CHARACTERISTICS
Highlighted lesion codes indicate lesions from a single patient represented by a single color.
Tumor risk factors highlighted in purple, patient high risk factors highlighted in lilac. EGFR trafficking status is
designated as Internalized (I), Disregulated (D) or Not visualized (N).

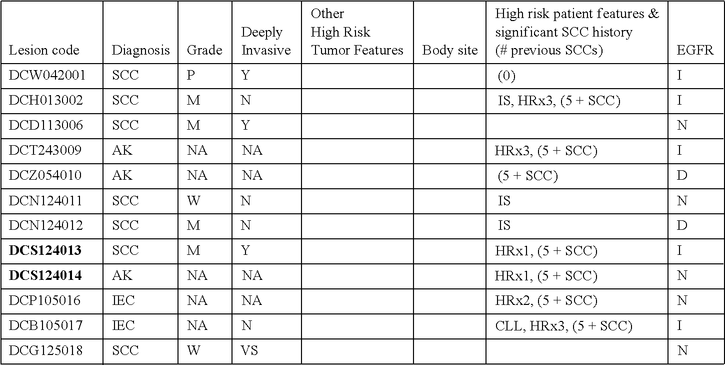

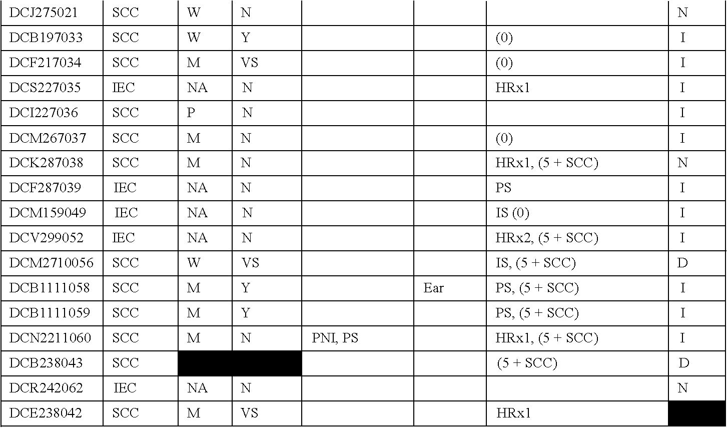

HR = High Risk SCC, Ag = Aggressive SCC, PNI = Perineural Invasion, IS = Immunosuppressed, PS = Possibly recurrent SCC (Not taken as independent evidence of HR unless confirmed)

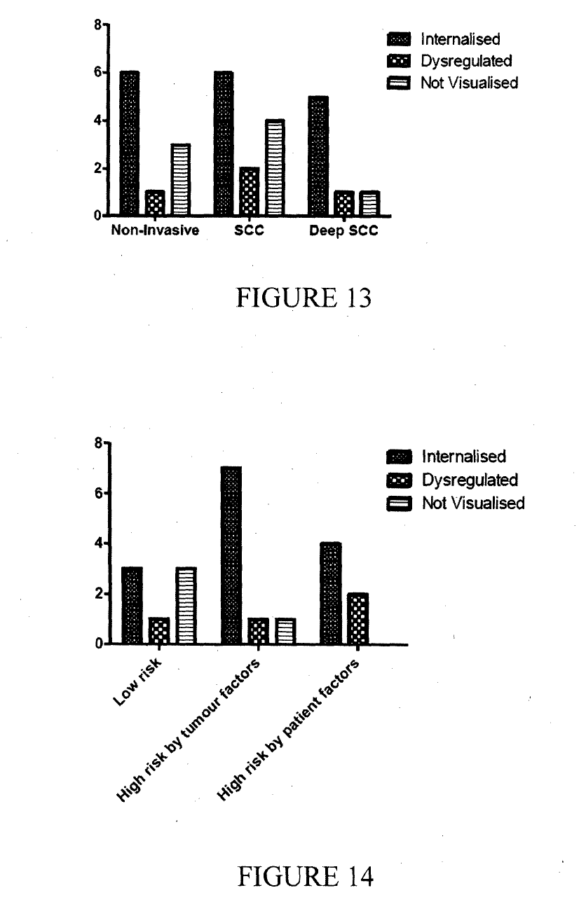

One AK out of three and three SCCs out of 19 were of the disregulated phenotype (FIG. 11). None of the seven IECs were disregulated, with five internalizing and two not visualized. Five of the SCCs were also in the not visualized category. Most tumors were classified as moderately differentiated. Most of the samples which could not be categorized as endocytosing or not were in the well differentiated grade tumors (FIG. 12). All the poorly differentiated tumors could be classified. This may relate to either EGFR expression or other particular features of the tumor tissue. Of the three disregulated SCCs, each was in a tumor of a different grade. Of 12 SCCs which were not reported as invasive to deep levels in histology, two were disregulated while one of seven deeply invasive SCCs was disregulated (FIG. 13). Approximately half (15/29) of the tumors could be classified as ‘high risk’ based on either tumor or patient factors (FIG. 14). Only one (25%) of the disregulated tumors was high risk by tumor factors as opposed to eight of the 17 internalizing lesions (47.1%). However, half (2) of the disregulated tumors occurred in high risk patients as opposed to just 23.5% (4) of the internalizing lesions.

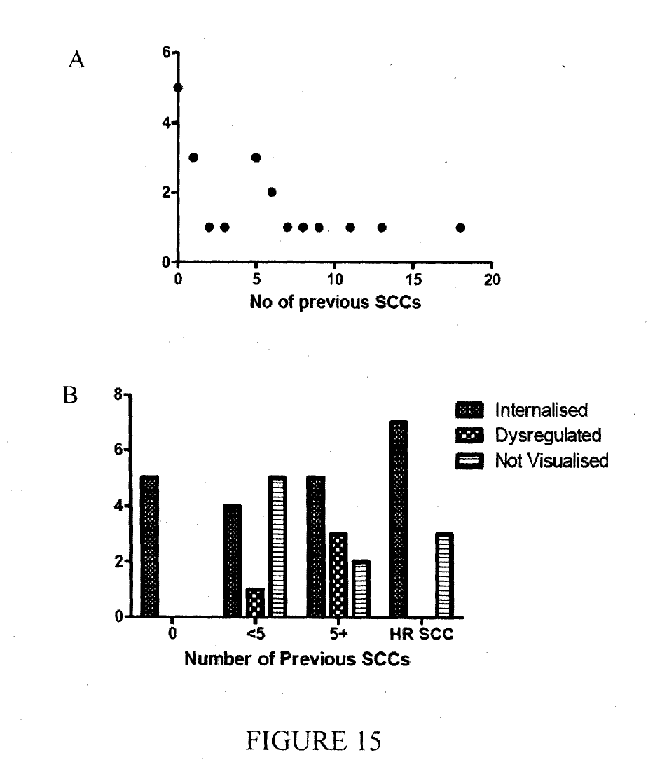

When SCC history was assessed, the data lent itself to division into three subgroups based on peaks in the numbers of previous lesions (FIG. 15A). Previous IEC and AK were not considered as these data are extremely difficult to obtain. None of the disregulated tumors occurred in patients who had no previous SCCs. Three quarters of the disregulated tumors were found in patients with a history of five or more SCCs and none of the disregulated tumors occurred in patients who had a previous high risk SCC (FIG. 15B).

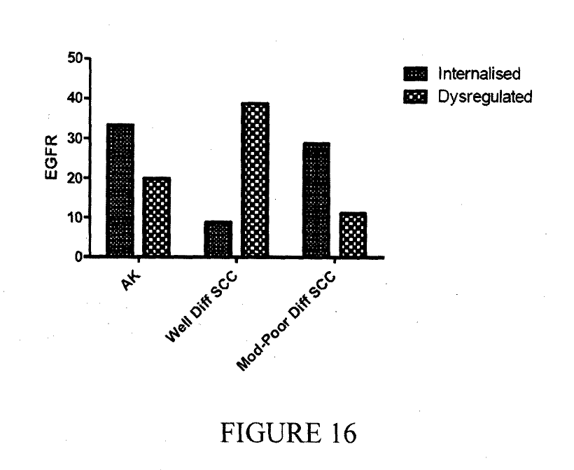

Digital analysis of EGFR total expression levels in basal and invasive regions of keratinocytes was performed on matched pairs of internalizing and disregulated tumors (FIG. 16). This included one example in each category of an AK, a well differentiated SCC and a moderately to poorly differentiated SCC. There was no correlation between trafficking status and total EGFR expression.

### Conclusions

A live human tissue EGF uptake study has been performed. Analysis has been performed by whole mount laser confocal fluorescent imaging. This technique can be used to determine trafficking status in early SCC and precursor lesions. Pathological features can be determined and dysplastic keratinocyte populations have been determined by subjective and objective measures. From this data set the following conclusions can be made:
Disregulation may be an early event in the development of SCC
Disregulation is more commonly seen in SCC with low risk characteristics)
Disregulation occurs in patients who have had previous SCC and is more commonly seen in patients with multiple previous SCCs
Disregulation occurs in patients who are at high risk for SCC
Disregulation is not dependent on EGFR total expression level.

It is proposed therefore that:
Disregulation may occur in high risk individuals with previous SCC and is associated with a preponderance of less aggressive tumors
Disregulation does not appear to correlate strongly with aggressive tumor characteristics and is independent of total EGFR expression level.

### Example 2

### EGRF Endocytosis is Disregulated in Human Squamous Cell Carcinoma

### Material and Methods

Cell Lines

Cell line identities were verified by SNP analysis. Cells were mycoplasma free and tested regularly. Cells were grown in Ham's F-12 media: DMEM supplemented with 10% FBS, 10 mM HEPES, 2 mM L-Glutamine, 1 mM Sodium pyruvate (Gibco, Invitrogen). Normal Human Embryonic Keratinocytes (HEKs) from neonatal foreskins were cultured as described in (11). HEK cultures were grown and maintained in low-calcium serum-free KC culture medium (Gibco, Invitrogen) supplemented with 2.5 μg EGF and 25 mg BPE. For basal conditions cells were grown for 3 hrs in complete media but without 10% FBS.

Antibodies

Primary Ab: AKT (C67E6; Cell. Signaling Technology), Clathrin (BF-06; EXBIO) EGFR (528; Cell Signaling Technology), EGFR (31 G7; Invitrogen), ERK2 (c-14; Santa Cruz), phospho-EGFR (Tyr1068, D7A5; Cell Signaling Technology), phospho-AKT (Ser473, D9E; Cell Signaling Technology), phospho-44/42 MAPK (ERK1/2, Thr202/Tyr204, E10, Cell Signaling Technology) and β-tubulin (2-28-33, Zymed). Secondary Ab: Alexa 488 goat anti-mouse IgG (A11001; Invitrogen), Alexa 594 donkey anti-rabbit IgG (A21207; Invitrogen), Alexa 594 goat anti-human IgG (A11014; Invitrogen), Alexa 594 goat anti-mouse IgG (A11005; Invitrogen), HRP-conjugated goat anti-mouse (F21453; Invitrogen), HRP-conjugated goat anti-rabbit (A10547; Invitrogen).

Fluorescent EGF Internalization Assay on Mouse Xenografts and Patient SCC Samples

Tumor samples were collected, with PA Hospital consent and ethics approval, on ice either from PAH Pathology (post-surgery) or from procedure room (biopsy). Samples were sliced into ˜1 mm sized pieces and washed three times for 10 min in basal media at 4° C. 20 ng/mL of EGF-Alexa₄₈₈ (E-13345; Invitrogen) was added to the final wash for 5, 15 or 30 min and placed at 37° C. In addition, sample was left untreated, treated with 25 μg/mL of Cetuximab (Erbitux; MerckSerono) for 30 min prior to addition of EGF for 15 mins or treated with 10 μg/mL Tfn-Alexa⁵⁵⁵ (Invitrogen, T-35352) or 10 μg/mL Dextran-Alexa₅₉₄ (Invitrogen, D-22913). Samples were washed five times at 4° C. in PBS+0.1% Triton X-100 (PBTX) for 30 min and then fixed in 4% PFA/PBS overnight (O/N) at 4° C. Samples were washed twice in PBS and placed in 100% MeOH at 4° C. for 2 hrs. Tissue autofluorescence was reduced using Dent's bleach (4 MeOH: 1 DMSO: 1 30% H₂O₂) (12) for 2 hrs at room temperature (RT). Samples were rehydrated using a methanol/PBTX series. Samples were incubated with DAPI (50 mM) for 10 min, washed for 2 hrs with PBTX, washed twice in PBS, rinsed with H₂0 and mounted in Prolong Gold (Invitrogen) on concave microscope slides. Images were acquired using a Zeiss 510 Meta confocal with a 63× objective with Zen 2008 software.

Whole Mount Immunofluorescence (IF) on Patient SCC Samples

Whole Mount samples were processed as described above however following bleaching step, samples were blocked for 4 hrs in 10% horse serum/PBTX and incubated with primary Ab in block O/N at 4° C. Following 5×20 min washing in PBTX, the corresponding Alexa Fluor 594 secondary Ab in block was applied for 1 hr at RT. Samples were incubated with DAPI (50 mM) for 10 min, mounted and imaged as above.

Section IF and H&E Staining

Formaldehyde fixed tumor samples were paraffin embedded and sectioned at 4 μm. Sections were incubated in a heating oven (56° C.) for 1 hr prior to rehydration. For IF, sections were xylene treated and rehydrated through a standard alcohol series. Following antigen retrieval using pH 9.0 Tris-EDTA buffer sections were blocked in 4% Horse Serum/1% BSA/PBS for 1 hr at RT and primary Ab incubated O/N at 4° C. Following washes in block the Alexa fluor 488 secondary Ab was added for 1 hr at RT. After washing sections were DAPI stained and mounted as described above. Images were acquired using a Zeiss 510 Meta confocal with a 25× objective with Zen 2008 software. A standard H&E staining protocol was performed by Pathology Queensland at PA Hospital and images acquired on a Nikon Eclipse 50i microscope using a 4× or 20× objective.

Quantitative EGF Internalization Assay

The SCC cell lines were plated in 100 mm dishes at 80% confluency. A modified version of ligand internalization assay as described in (13) was performed to measure EGF internalization. After basaling cells were treated with 1 ng/mL of Biotin-EGF (E3477; Invitrogen) at 37° C. for 0, 5, 15 or 30 min. Internalization was stopped by placing cells on ice and washing with ice cold PBS. Non-internalized and membrane bound Biotin-EGF was blocked by washing with 1 μg/mL avidin (Sigma) followed by quenching with 10 μg/mL Biotin (Sigma). A non avidin-biotin blocked control was included to determine total EGF levels at 15 min. Cells were then washed three times in cold PBS and lysed in 150 μL of RIPA buffer containing protease and phosphatase inhibitors (Calbiochem). EGF levels were measured using Human EGF ELISA Kit (KHG0061; Invitrogen) with some modifications. Protein lysate (10 μg) in sample diluent and a two-fold dilution series (1 ng/ml to 15.6 pg/mL) of Biotin-EGF (standard curve) were plated onto a human EGF coated 96-well plate and incubated for 2 hrs. Following washes, streptavidin-HRP was added and the plate incubated for 30 min at RT. Stabilized Chromogen was added after washing and the plate incubated in dark. After 30 min a Stop solution was added to each well and absorbance was read at 450 nm. The assay was performed three times in technical duplicate.

Fluorescent EGF/Tfn Internalization Assay

Cells were plated at 80% confluency on coverslips. Following basaling cells were treated with 10 ng/mL of EGF-Alexa⁴⁸⁸ or 25 μg/ml TFN-Alexa⁵⁵⁵ at 37° C. for 5, 15 or 30 min or left untreated. Cells were washed three times in cold PBS and fixed in 3% PFA. After further washing cells were DAPI stained and mounted onto glass slides and imaged using a Zeiss 510 Meta confocal with a 63× objective.

EGFR Level ELISA Assay

SCC cell lines and HEKs were plated in 100 mm dishes. Cells were washed three times in cold PBS and lysed in 1501.11 of RIPA buffer containing protease and phosphatase inhibitors (Calbiochem). EGFR levels were measured from 12-15 μg of total protein lysate using STAR EGFR ELISA Kit (Millipore). The assay was performed three times in technical duplicate. An unpaired Student's T-test was performed to compare levels of EGFR of each SCC lines compared to HEKs using GraphPad Prizm 5.

Immunoblotting

SCC cell lines were plated in 100 mm dishes at 80% confluency. After basaling cells were treated with 1 ng/mL of EGF-Alexa⁴⁸⁸ at 37° C. for 5, 15 or 30 min or left untreated (control and EGFR). Cells were then washed three times in cold PBS and lysed in 4 ml lysis buffer [50 Mm Tris-HCl pH7.5, 100 mM NaCl, 1% Triton X-100, protease and phosphatase inhibitors (Calbiochem)]. For immunoblotting equal concentrations, as determined by BCA assay (Pierce), of protein lysates were separated by SDS-PAGE, semi-dry transferred to PDVF membranes (Millipore). Membranes were blocked in 2% BSA in TBS+0.1% Tween-20 (TBST) and probed with primary Ab 0/N at 4° C. and then washed with TBST three times for 10 min. The membranes were incubated with corresponding secondary Ab in block for 1 hr. After washing the membranes were incubated with ECL (1:1 of Supersignal West Pico and Supersignal Dura; Thermo Scientific) and exposed to film (FUGI). Each experiment was performed three times.

### Results

Ligand-dependent receptor endocytosis has not previously been examined in viable human tumors before. Therefore, the present inventors sought to develop a method in which one could image, in real time, EGFR endocytosis in viable human tumors. Previous studies have characterized EGFR endocytosis in established cell line models in two dimensional tissue culture systems (14). However, there are reports suggesting that the biology of receptor trafficking observed in vitro may not reflect receptor trafficking in vivo (15). In vivo, tumor cells exist within a three-dimensional micro-environment surrounded by non-transformed cells and connective tissue elements. Interactions between tumor cells and their stromal environment (cellular and non-cellular) will dictate cellular behavior and will likely influence receptor biology. Therefore, the present inventors developed a method to examine ligand-dependent EGFR endocytosis in ex vivo samples of fresh living human tumors.

Ligand-Stimulated EGFR Uptake can be Visualized in Live Xenograft Tissue

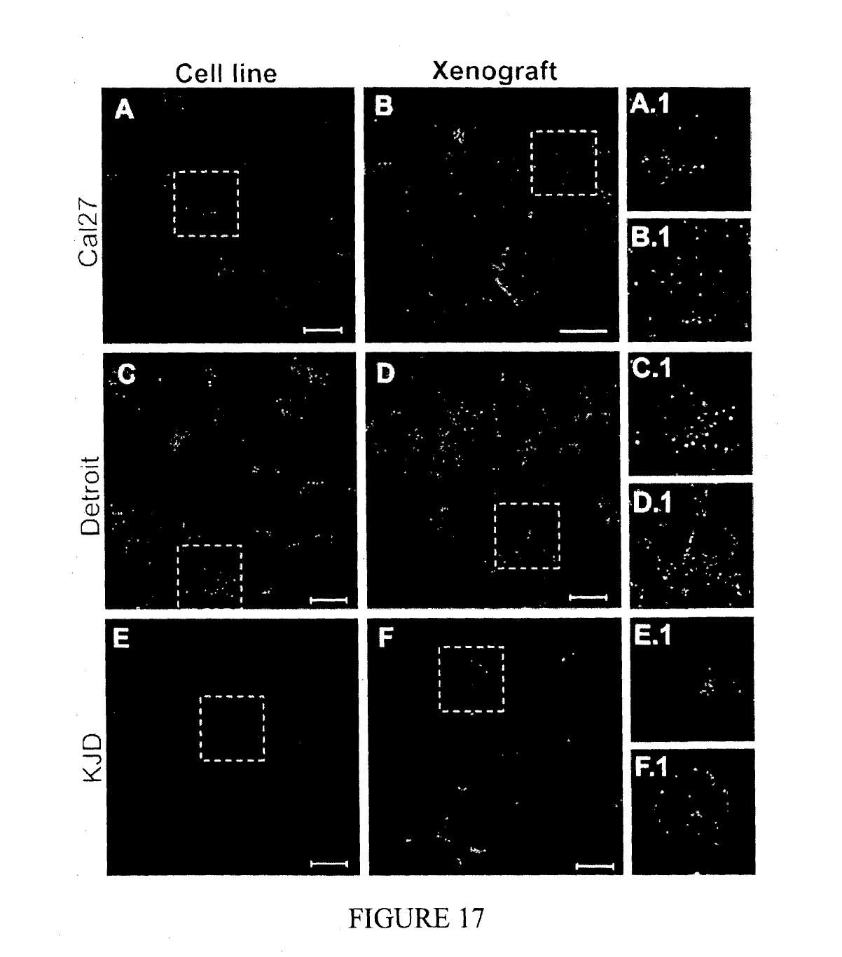

Prior to using human tumors the present inventors optimized their methodology using xenograft models of established SCC cell lines injected into NOD/SCID mice (16). Tumors were removed and processed as described in Materials and Methods for this example. After uptake the tissue was fixed and bleached as described. The samples were then mounted in microscopy wells and analyzed by confocal microscopy (FIG. 17). All xenograft tumors demonstrated specific EGF binding and varying degrees of receptor internalization at 15 minutes in vitro and in vivo (FIG. 17). The xenograft tumors showed similar uptake to their monolayer cultured counterparts.

EGFR Ligand-Induced Endocytosis is Disregulated in Approximately 37% of Human SCC

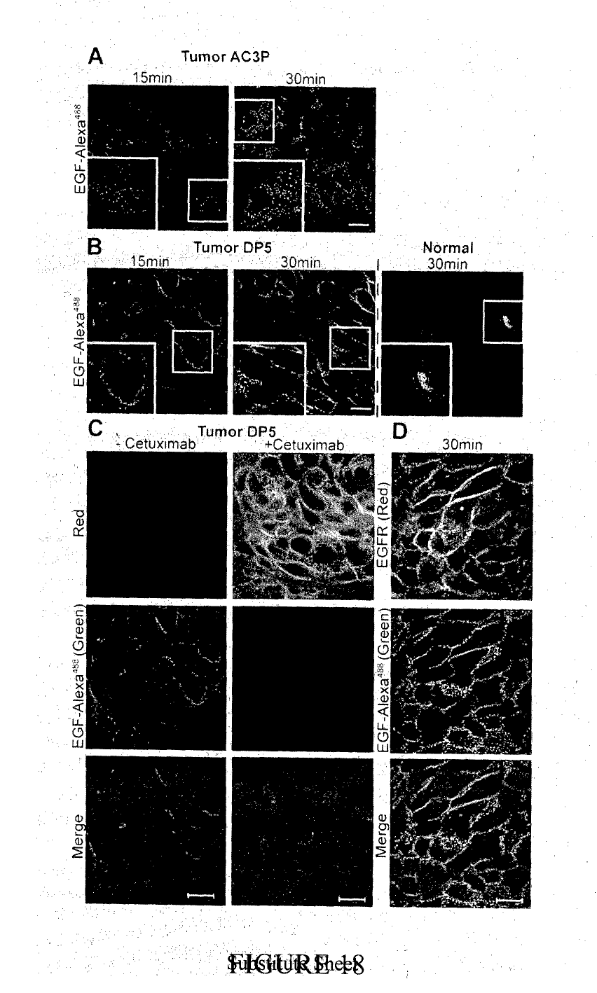

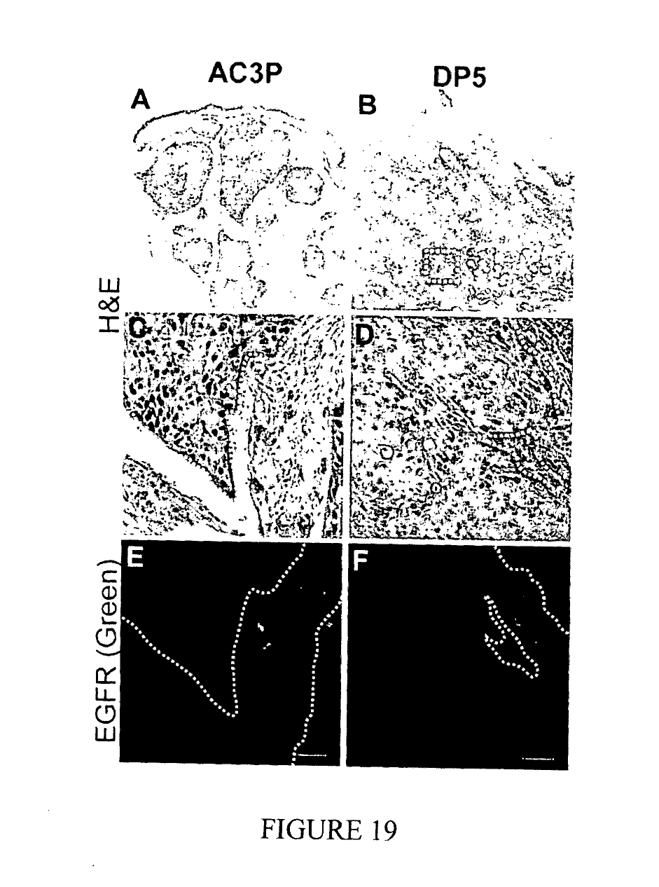

Having validated their method in xenografts, the present inventors analyzed the ligand-dependent EGFR internalization in human tumors (FIG. 18). They examined eight viable SCC tumor samples excised at surgery or obtained from core biopsies. In these embodiments, tissue should be non-frozen, non-fixed and non-necrotic for uptake to occur. In three of the eight patients uptake of EGF into endosomal structures was observed, as shown for patient AC3P (FIG. 18A). Five of the eight tumors show plasma membrane binding of EGF but little internalization (FIG. 18B, Table 4), however after 30 minutes internalization is observed in a small subpopulation of cells (approx. 2-5% of total tumor cells). The lack of internalization cannot be attributed to the loss of viability of the patient samples since the present inventors were able to show both Dextran uptake and/or transferrin receptor (TfnR) internalization in these same samples. Non-viable samples showed no binding or uptake above tissue autofluorescent levels. Controls included no EGF addition and 4° C. To further validate the specificity of EGF fluorescence tumor samples were pre-incubated with high concentrations of Cetuximab, the monoclonal antibody (Ab) which binds to the ligand-binding domain of EGFR and is used in SCC therapy. It was found that prior binding of Cetuximab prevented EGF-Alexa⁴⁸⁸ binding and uptake (FIG. 18C). Post-fixation labeling of the samples with anti-EGFR showed co-localization with the EGF-Alexa⁴⁸⁸ (FIG. 18D). Finally, to confirm that the EGF binding and uptake observed occurred only in the dysplastic/tumorigenic cells, serial sections of untreated tissue (formalin fixed on removal) samples for, patients AC3P and DP5 were stained with Hematoxylin and Eosin (H&E) or fluorescently labeled anti-EGFR Ab. EGFR staining was demonstrable to detectable levels only in the dysplastic cells, as shown by the H&E staining (FIG. 19).

TABLE 4
TUMOR CHARACTERISTICS

| ^{a}ID | ^{b}Site | ^{c}EGF |
|---|---|---|
| RW2 | Tonsil | Plasma membrane |
| JT3 | Tongue | Plasma membrane |
| DP5 | Larynx | Plasma membrane |
| EG7 | Tonsil | Internalized |
| BV8 | Oral Cavity | Plasma Membrane |
| SW1P | Cutaneous | Internalized |
| AC3P | Cutaneous | Internalized |
| JM2P | Cutaneous | Plasma membrane |

^{a}De-identified patient code
^{b}Site from which tumor was excised
^{c}Status of ligand-induced EGFR endocytosis after EGF stimulation

### EGFR Internalization in SCC Cell Lines is Disregulated in the Initial Receptor Concentration and Recruitment Stages of Endocytosis

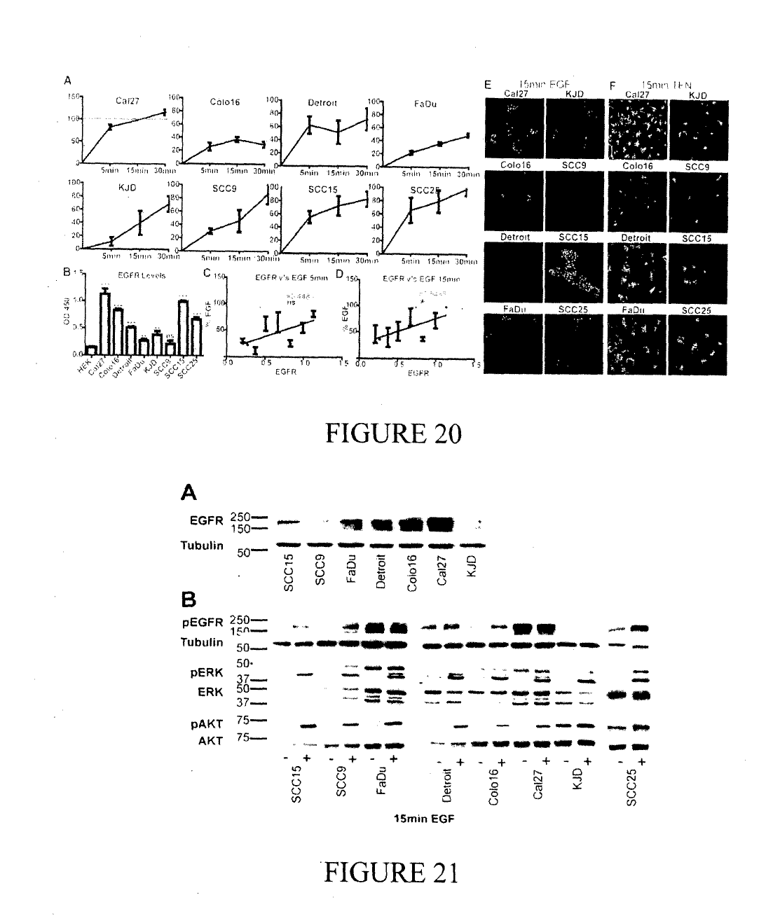

Next, the present inventors examined whether there was a relationship between ligand-dependent EGFR trafficking status and EGFR expression levels or ligand-induced signal transduction. Both human SCC samples as well as established human SCC cell lines were shown to display varying degrees of ligand-dependent EGFR internalization dysfunction (FIGS. 17 and 18). Thus, the SCC cell lines are a useful model to interrogate the relationship between EGFR internalization dysfunction and EGFR expression or signaling activity. Low concentrations of recombinant EGF ligand were used to ensure that uptake was mediated via clathrin-coated pits (17). For each of the indicated cell lines the internalization of biotinylated EGF was measured using standard endocytosis assays as described (Materials and Methods for Example 2). Receptor internalization, as a percentage of total EGF binding at 15 minutes, was measured over a 30 minute time-course for each cell line (FIG. 20A). Fifteen minutes was chosen as the time-point for measurement of total binding as after this time-point degradation of the receptor increases. Ligand-stimulated EGFR internalization in the first five minutes provides an estimate of the initial rate of ligand binding and the efficiency with which ligand binding stimulates receptor endocytosis. In contrast, internalization measured at 15 minutes reflects the relative capacity of the cells to bind and internalize the EGFR. The eight human SCC cell lines varied considerably in their ability to bind and internalize ligand within five minutes and interestingly showed less disregulation than the human tumor samples, reflecting previously observed differences between cell line responses in culture as opposed to xenograft or tumor models (18).

### Disregulation of EGFR Internalization does not Correlate in the Initial Stages with EGFR Expression Level

EGFR expression level in each cell line was measured by ELISA assay (FIG. 20B). There was no correlation between EGFR expression and initial rates (5 min) of EGF-dependent receptor internalization (FIG. 20C). However, there was an association between EGFR expression levels and the total capacity (15 min) of the cell lines to internalize EGFR (FIG. 20D). EGF-Alexa⁴⁸⁸ was added to the cells on coverslips as for Biotin-EGF in biochemical experiments. Time points were fixed and imaged. Immunofluorescence uptake in each cell line correlated with the assay levels of uptake (FIG. 20E). Thus, initial rate constants for ligand binding and internalization appear to be independent of receptor number suggesting that proximate effects associated with coupling of ligand binding and internalization may be disregulated within SCCs.

### Disregulation does not Correlate with any Single Signaling Defect but does Correlate with Pathway Defects

The uncoupling of EGFR expression and initial internalization rates is suggestive of defects in ligand-induced recruitment into clathrin coated vesicles (CCVs) or disregulation of CCV formation. However, analysis of TfnR internalization showed normal uptake suggesting the defect in endocytosis is specific to the ligand-induced uptake of EGFR in the SCC cell lines (FIG. 20F). These data show that the disregulation is not a global defect in ligand-dependent receptor endocytosis. Defects in ligand-dependent receptor endocytosis would be expected to be reflected in defects in ligand-induced signaling events. Therefore, all cell lines were examined by Western analysis for signaling output (FIG. 21). Cells were stimulated with EGF as described for the endocytosis assays above over the same time-course. Three biological replicates were probed for each time-point and for each cell line. Lysates were probed for EGFR, phospho-EGFR, tubulin, total ERK, phospho-ERK, total AKT and phospho-AKT (FIG. 21). Although large variability in signaling between cell lines was observed, no correlation between this and EGFR levels was observed and no single signaling alteration correlated with endocytic defects.

Analysis indicated no clear relationship between constitutive phosphorylation of the EGFR and EGFR internalization rate or capacity (FIG. 21). There was no association between the ability of ERK and AKT to respond to EGF binding and EGFR internalization rates or capacity (FIG. 21). There was no correlation between the expression of total AKT or ERK levels and EGFR internalization rates or capacity (FIG. 21). A, similar relationship was observed in the xenograft samples examined by immunohistochemistry for phospho-AKT. These data suggest that ligand-dependent EGFR internalization is not due to a single defect in ligand-stimulated EGFR phosphorylation or ligand-coupled ERK and AKT signaling.

However, each cell line which showed EGFR internalization changes also had one or more alterations in signaling; thus changes in EGFR internalization may be an overall biomarker for signaling pathway disruption due to the intricate feedback mechanisms.

### Disregulation of EGF Internalization can be Visualized Using Post-Fixation EGFR Labeling of Pre-Stimulated Tissue

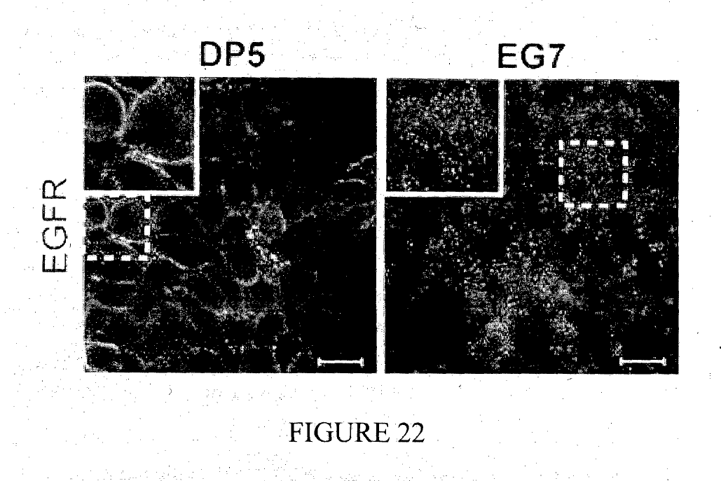

Finally, the present inventors examined whether receptor trafficking defects in tumor samples can be visualized in normal histopathological specimens. In particular, they tested post-fixation labeling of tumor samples stimulated with EGF for 15 minutes, processed the tissue as described in materials and methods for the present example and labeled the sample with anti-EGFR Ab (FIG. 22). Stimulation was carried out simply by adding EGF to fresh tumor samples in serum free media. Localization of the EGFR, as observed by post-fixation labeling, reflected the results the present inventors had observed with direct imaging of the EGF ligand (FIGS. 18A and B). Thus, after stimulation, labeling of the EGFR can also be used to classify SCC as endocytosis competent or disregulated with the advantage of increased fluorescence from secondary Ab labeling as opposed to direct labeling of the EGF.

### Discussion

Substantial international effort has gone into proteomics, genomics and transcriptomics to try to determine a correlation between patient response to anti-EGFR therapy and tumor biology. However, it has not been possible to characterize the trafficking and spatiotemporal regulation of receptors in human tumors. The present inventors have developed a method to investigate ligand binding and receptor-mediated endocytosis of EGF/EGFR in human tumor samples in an ex vivo context. They have demonstrated that EGFR endocytosis is frequently disregulated in human SCC. Interrogation of human SCC cell lines also revealed disregulation of EGFR endocytosis which appeared to impact the initial stages of ligand-induced recruitment of EGFR. Finally, EGFR endocytic dysfunction showed no single correlation with EGFR expression level, EGFR phosphorylation or with the activation of downstream effectors such as AKT or ERK but EGFR endocytic disregulation may be a biomarker for overall signaling dysfunction. This now enables one to examine the potential relationship between EGFR trafficking and prognosis, tumor progression and EGFR inhibitor resistance and acquired resistance in epithelial tumors.

In particular, the present inventors have found that in 37% of the human SCC tumors EGFR is not endocytosed in response to ligand induction. Breakdown of endocytosis in the oncogenic activation of receptor tyrosine kinases (RTKs) has been reviewed in detail recently (19) and has been highlighted in a recent report showing enhancement of tumorigenesis via RTK MET is dependent on its endocytic rates and localization (20). In cells where EGFR levels are enriched at the plasma membrane EGF stimulation results in sustained activation of both MAPK and PI3K pathways (19). Under hypoxic conditions many RTKs exhibit prolonged activation and while the mechanism for this is unknown the effect is increased oncogenesis (21). Thus it may be that inhibition of EGFR internalization is a mechanism of oncogenesis or progression.

The instant analysis of endocytosis in SCC cell lines shows that the main disregulation of EGFR uptake occurs in the initial five minutes after ligand addition. This fits with the inventors' observations in human tumors where receptor-mediated internalization of EGFR from the plasma membrane does not occur. The initial stage of stimulation corresponds to the time taken for recruitment of activated EGFR from membrane domains to the CCVs and encompasses EGFR oligomerization, ubiquitination, alterations in cortical actin and the diffusion of EGFR away from lipid-rich domains. Consistent with this, no alteration in the internalization of the constitutively endocytosed TfnR was found, suggesting that clathrin-mediated endocytosis remains functionally intact.

The present analysis of EGFR expression level and signaling in SCC cell lines indicates that endocytic disregulation does not correlate with EGFR expression level in the recruitment phase. Cells with normal endocytosis also tended to show normal signaling outputs while cells with disregulated internalization had disrupted signaling output. However, no single signaling output could be correlated to endocytic disregulation. Increased expression of other Erb-B receptor family members and changes in cholesterol levels leading to increased lipid rafts would also affect EGFR retention on the plasma membrane. As therapies are directed against the EGFR itself, this alteration in localization and exposure of the EGFR will likely impact therapeutic response (22) and it may be that diagnosis of the endocytic capacity of EGFR in response to ligand binding in epithelial cancer will function as an overall biomarker for a number of intracellular changes leading to alteration in therapy response or tumor progression.

### Example 3

### Analysis of EGFR Trafficking by Three Dimensional Structured Illumination (Super Resolution) Microscopy (3D-SIM)

### Materials & Methods

Super-Resolution Microscopy

For three dimensional structured illumination microscopy (3D-SIM), images were captured on a Deltavision OMX V3 Imaging System (Applied Precision), EMCCD cameras (CascadeII 512×512 Photometrics) and using a 60×1.4-NA UPlanSApo oil-immersion objective (Olympus) with oil of a refractive index of 1.524. Images were acquired with a Z-step 0.125 μm with 23-53 steps over thickness of 3-6.5 μm at a laser power of 10%. Images were computationally reconstructed using Deltavision SoftWorX6.0 Beta19 (Applied Precision).

### Results

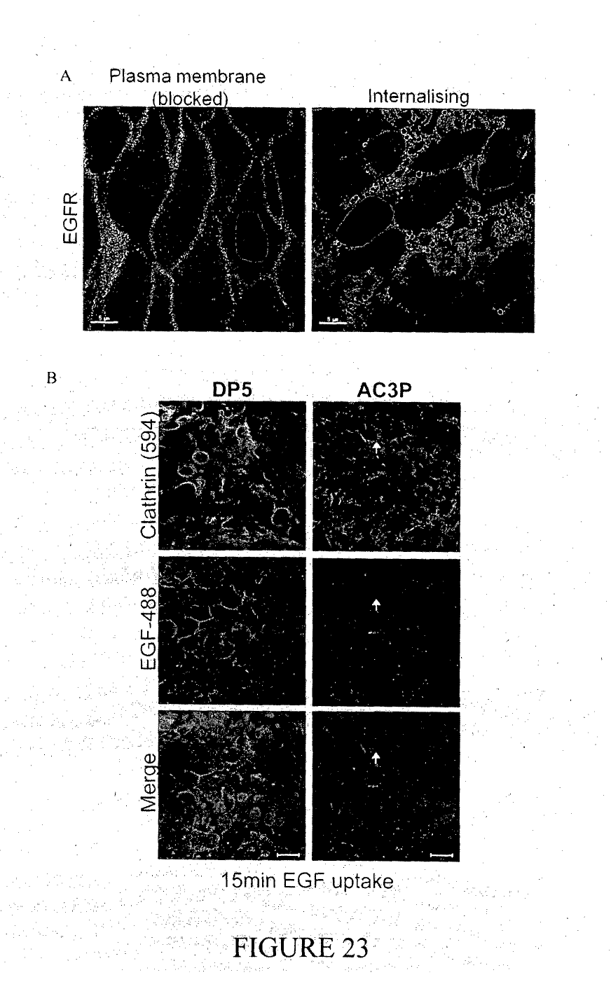

The present inventors also analyzed tumor samples by three dimensional structured illumination (super resolution) microscopy (3D-SIM) to more clearly show the EGFR trafficking differences within patient samples. FIG. 23A shows a patient whose EGFR does not undergo EGF-induced internalization and only plasma membrane localization can be observed while FIG. 23A also shows a normally internalizing EGFR patient, where the human cell endosomes can be clearly observed. Co-localization of EGFR with clathrin (FIG. 23B) demonstrated that in patient samples which failed to internalize EGFR, clathrin recruitment to the membrane was decreased in response to EGFR ligand stimulation and appeared to have increased distribution to the Trans-Golgi Network.

### Summary

In summary, the present inventors describe herein a novel imaging assay to monitor EGF-induced EGFR internalization in living human tumor samples. They show that human SCCs can be categorized as either EGFR trafficking-competent or EGFR trafficking-incompetent. In addition, they show that EGFR trafficking status can contribute to EGFR plasma membrane expression levels, which in turn is predictive of cetuximab-induced ADCC of the target SCC cells. Based on these findings it is predicted predict that patients whose EGFR receptor is trapped on the plasma membrane will respond best to monoclonal antibody therapy.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Wheeler D L, Dunn E F, & Harari P M (2010) Understanding resistance to EGFR inhibitors-impact on future treatment strategies. Nature Reviews Clinical Oncology 7(9):493-507.
2. Avraham R & Yarden Y (2011) Feedback regulation of EGFR signalling: decision making by early and delayed loops. Nat Rev Mol Cell Biol 12(2):104-117.
3. Bonner J A, et al. (2006) Radiotherapy plus cetuximab for squamous-cell carcinoma of the head and neck. New Engl J Med 354(6):567-578.
4. Montagut C, et al. (2012) Identification of a mutation in the extracellular domain of the Epidermal Growth Factor Receptor conferring cetuximab resistance in colorectal cancer. Nat Med 18(2):221-223.
5. Ang M K & Hayes D N (2010) Imaging: Mass spectrometry in HNSCC—a peek at response prediction? Nat Rev Clin Oncol 7(4):193-195.
6. Gumuskaya B, et al. (2010) EGFR expression and gene copy number in triple-negative breast carcinoma. Cancer Genet Cytogenet 203(2):222-229.
7. Saridaki Z, Georgoulias V, & Souglakos J (2010) Mechanisms of resistance to anti-EGFR monoclonal antibody treatment in metastatic colorectal cancer. World J Gastroenterol 16(10):1177-1187.
8. Martinelli E, De Palma R, Orditura M, De Vita F, & Ciardiello F (2009) Anti-epidermal growth factor receptor monoclonal antibodies in cancer therapy. Clin Exp Immunol 158(1):1-9.
9. Siena S, Sartore-Bianchi A, Di Nicolantonio F, Balfour J, & Bardelli A (2009) Biomarkers predicting clinical outcome of epidermal growth factor receptor-targeted therapy in metastatic colorectal cancer. J Natl Cancer Inst 101(19):1308-1324.
10. Moroni M, et al. (2005) Somatic mutation of EGFR catalytic domain and treatment with gefitinib in colorectal cancer. Ann Oncol 16(11):1848-1849.
11. Moroni M, et al. (2005) Gene copy number for epidermal growth factor receptor (EGFR) and clinical response to antiEGFR treatment in colorectal cancer: a cohort study. Lancet Oncol 6(5):279-286.
12. Lievre A, et al. (2006) KRAS mutation status is predictive of response to cetuximab therapy in colorectal cancer. Cancer Res 66(8):3992-3995.
13. Di Nicolantonio F, et al. (2008) Wild-type BRAF is required for response to panitumumab or cetuximab in metastatic colorectal cancer. J Clin Oncol 26(35):5705-5712.
14. Vieira A V, Lamaze C, & Schmid S L (1996) Control of EGF receptor signaling by clathrin-mediated endocytosis. Science 274(5295):2086-2089.
15. Rudnick S I, et al. (2011) Influence of affinity and antigen internalization on the uptake and penetration of Anti-HER2 antibodies in solid tumors. Cancer Research 71(6):2250-2259.
16. Cameron S R, et al. (2010) Tumor-initiating activity and tumor morphology of HNSCC is modulated by interactions between clonal variants within the tumor. Lab Invest 90(11):1594-1603.
17. Sigismund S, et al. (2008) Clathrin-mediated internalization is essential for sustained EGFR signaling but dispensable for degradation. Dev Cell 15(2):209-219.
18. Matsuo T, et al. (2011) Analysis of the anti-tumor effect of cetuximab using protein kinetics and mouse xenograft models. BMC Res Notes 4(1):140.
1.9. Abella J V & Park M (2009) Breakdown of endocytosis in the oncogenic activation of receptor tyrosine kinases. Am J Physiol Endocrinol Metab 296(5):E973-984.
20. Joffre C, et al. (2011) A direct role for Met endocytosis in tumorigenesis. Nat Cell Biol.
21. Franovic A, Holterman C E, Payette J, & Lee S (2009) Human cancers converge at the HIF-2alpha oncogenic axis. Proceedings of the National Academy of Sciences of the United States of America 106(50):21306-21311.
22. Spangler J B, et al. (2010) Combination antibody treatment down-regulates epidermal growth factor receptor by inhibiting endosomal recycling. Proc Natl Acad Sci USA 107(30):13252-13257.

1. A method for classifying an EGFR positive tumor into a subtype selected from an EGFR antagonist sensitive subtype or an EGFR antagonist resistant subtype, the method comprising, consisting or consisting essentially of analyzing the ligand-induced EGFR internalization status of the tumor, wherein an impaired or abrogated ligand-induced EGFR internalization status indicates that the tumor is an EGFR antagonist sensitive subtype and wherein an unimpaired ligand-induced EGFR internalization of status indicates that the tumor is an EGFR antagonist resistant subtype.

2. A method according to claim 1, wherein the ligand-induced EGFR internalization status is analyzed in the absence of analyzing KRAS status of the tumor.

3. A method according to claim 1, wherein the ligand-induced EGFR internalization status is analyzed in the absence of analyzing BRAF status of the tumor.

4. A method according to claim 1, wherein the ligand-induced EGFR internalization status is analyzed in the absence of analyzing molecules involved in EGFR-associated downstream signaling.

5. A method according to claim 1, wherein an impaired or abrogated ligand-induced EGFR internalization is indicated when, after at least 10 minutes in the presence of an EGFR ligand, at least 90% of the EGFR in cells of the tumor is localized or remains localized to the plasma membrane of the cells.

6. A method according to claim 1, wherein an unimpaired ligand-induced cell surface antigen internalization is indicated when, after at least 10 minutes in the presence of a ligand to the cell surface antigen: (a) less than 90% of the cell surface antigen in cell surface antigen-expressing cells of the tumor is localized or remains localized to the plasma membrane of the cells.

7. A method according to claim 1, wherein the EGFR positive tumor is selected from pre-cancerous, non-metastatic, metastatic, and cancerous tumors.

8. A method according to claim 7, wherein the EGFR positive tumor is associated with a cancer selected from carcinoma, lymphoma, blastoma, sarcoma, neuroendocrine tumors, mesothelioma, schwannoma, meningioma, adenocarcinoma, melanoma, leukemia, and lymphoid malignancies.

9. A method according to claim 8, wherein the cancer is selected from lung cancer, hepatocellular cancer, gastric or stomach cancer, pancreatic cancer, glioblastoma, cervical cancer, ovarian cancer, liver cancer, bladder cancer, hepatoma, breast cancer, colon cancer, rectal cancer, colorectal cancer, endometrial and uterine carcinoma, salivary gland carcinoma, kidney cancer, prostate cancer, vulval cancer, thyroid cancer, hepatic carcinoma, anal carcinoma, penile carcinoma, testicular cancer, esophageal cancer, tumors of the biliary tract, and head and neck cancer. In specific embodiment, the tumor is of an epithelial origin, non-limiting examples of which include cancer of the lung, colon, prostate, ovary, breast, and skin.

10. A method according to claim 1 wherein the EGFR positive tumor is a tumor is of an epithelial origin.

11. A method according to claim 10, wherein the EGFR positive tumor is selected from cancer of the lung, colon, prostate, ovary, breast, and skin.

12. A method according to claim 10, wherein the EGFR positive tumor is squamous cell carcinoma (SCC).

13. A method for stratifying a subject with an EGFR positive cancer into a treatment subgroup selected from responder to an EGFR antagonist and non-responder to an EGFR antagonist, the method comprising, consisting or consisting essentially of classifying an EGFR positive tumor according to the method of claim 1, and identifying the subject as a responder to an EGFR antagonist if an EGFR positive tumor of the subject is analyzed as having an impaired or abrogated ligand-induced EGFR internalization status or identifying the subject as a non-responder to an EGFR antagonist if an EGFR positive tumor of the subject is analyzed as having an unimpaired ligand-induced EGFR internalization status.

14. A method according to claim 13, further comprising obtaining a tumor sample from the subject for the analysis.

15. A method for treating a subject with an EGFR positive cancer, the method comprising, consisting or consisting essentially of stratifying the subject into a treatment subgroup selected from responder to an EGFR antagonist and non-responder to an EGFR antagonist according to the method of claim 13, and administering an EGFR antagonist to the subject on the basis that the subject is stratified into the responder subgroup or administering a cancer therapy other than an EGFR antagonist to the subject on the basis that the subject is stratified into the non-responder subgroup.

16. A method according to claim 15, wherein the EGFR antagonist is selected from anti-EGFR antibodies and anti-EGFR tyrosine kinase inhibitors.

17. A kit comprising a reagent for use in the method of claim 1.

18. The kit of claim 17, wherein the reagent is a ligand of EGFR.

19. The kit of claim 18, wherein the reagent is an antibody that binds to EGFR.

20. The method of claim 1, wherein all or a part of the method is performed by a processing system.
